(19) United States
(12) Patent Application Publication
(10) Pub. No.: US 2012/0093896 A1
(43) Pub. Date: Apr. 19, 2012

(54) USE OF NANODISPERSIONS TO PROTECT WATER-SOLUBLE INGREDIENTS IN COSMETIC END FORMULATIONS

(75) Inventors: Sébastien Mongiat, Sierentz (FR); Julie Grumelard, Huningue (FR); Werner Baschong, Basel (CH); Bernd Herzog, Grenzach-Wyhlen (DE)

(73) Assignee: CIBA CORPORATION, Tarrytown, NY (US)

(21) Appl. No.: 12/669,972

(22) PCT Filed: Jul. 22, 2008
(86) PCT No.: PCT/EP08/59568

§ 371 Date: Aug. 11, 2010

(30) Foreign Application Priority Data
Jul. 31, 2007 (EP) 07113510.7

(51) Int. Cl. A61K 8/04; A61K 8/66; A61K 8/02; A61Q 19/00; A61Q 5/00; A61K 8/67; A61Q 1/12; B82Y 40/00; B82Y 99/00

(52) U.S. Cl. 424/401; 514/474; 424/94.4; 424/47; 424/69; 424/70.1; 977/926; 977/840; 977/788

(57) ABSTRACT

The invention describes the use of a nanodispersion, which comprises: a) a membrane-forming molecule, b) a co-emulsifier, c) a lipophilic component, and d) a water-soluble ingredient sensitive to oxidation in cosmetic end formulations, the nanodispersion being obtainable by (α) mixing the components (a), (b) and (c) until a homogeneous liquid is obtained (so-called nanodispersion pre-phase), and (β) adding the water or aqueous phase (discontinuous phase) which contains the water-soluble ingredient and optionally the non-aqueous polar organic solvent to the liquid obtained in step (α). Component (d) is preferably ascorbic acid or vitamin C.

The present invention relates to the use of a nanodispersion which comprises (a) a membrane-forming molecule, (b) a coemulsifier, (c) a lipophilic component, and (d) a water-soluble ingredient sensitive to oxidation, like for example ascorbic acid, in cosmetic end formulations such as for example sticks, gels, creams, lotions, milks, sprays, aerosols, foams, pastes, powders, lacquers pellets or make-ups.

Cosmetic preparations comprise, in addition to the basic substances responsible for forming the cosmetic formulations, other functional active agents. These are added to the cosmetic base formulations and are used, for example, for treatment, protection, colouring, cleansing, disinfection, for moisturising skin and for regenerating and activating skin or hair.

It is an object of the present invention to improve the stability of water-soluble ingredients against its own degradation/decomposition which is induced by oxygenous environment and also by water.

A preferred embodiment of this invention is based on ascorbic acid. Numerous patents and publications teach various ways of stabilizing ascorbic acid.

For example, EP-A-0 729 746 discloses an ascorbic acid delivery system comprising a two compartment kit. The first compartment is an anhydrous solution of ascorbic acid dissolved in a nonaqueous polar solvent such as a polyol. The second compartment is an alkaline aqueous solution. The contents of the two compartments are mixed immediately prior to use. The more alkaline aqueous solution increases the pH of the anhydrous ascorbic acid containing solution to provide a composition which does not tingle the skin.

U.S. Pat. No. 5,350,773 discloses stable liquid products used for liquid human and animal food. The compositions are in the form of anhydrous emulsions containing a nonpolar fatty material as the dispersed phase and a polar non aqueous solvent as the continuous phase.

U.S. Pat. No. 5,427,814 discloses a method for protecting a fat against oxidation by mixing ascorbic acid into apolar solvent, and adding the mixture to a heated mixture of tocopherol and lecithin.

U.S. Pat. No. 5,308,621 discloses a method for transdermal administration of ascorbic acid. Fine particulate ascorbic acid is suspended in a pharmaceutically acceptable carrier such as a polyol.

EP-A-0 755 674 discloses a stabilized ascorbic acid composition which contains a minimum level of water, in addition to a polyol and oil.

U.S. Pat. No. 5,587,149 discloses an ascorbic acid emulsion composition comprising ascorbic acid solubilised in a polyol as the dispersed phase and oil as the continuous phase.

However, none of the above patents teach a stable anhydrous ascorbic acid containing composition suitable for cosmetic application where high levels of ascorbic acid are solubilised in the composition and remain stable over a long period of time.

The present invention therefore provides the use a nanodispersion, which comprises
(a) a membrane-forming molecule,
(b) a coemulsifier,
(c) a lipophilic component, and
(d) a water-soluble ingredient sensitive to oxidation
   in cosmetic end formulations, the nanodispersion being obtainable by (α) mixing the components (a), (b) and (c) until a homogeneous liquid is obtained (so-called nanodispersion pre-phase), and (β) adding the water or aqueous phase (discontinuous phase) which contains the water-soluble ingredient and optionally the non-aqueous polar organic solvent to the liquid obtained in step (α).

Preferably, component (d) is ascorbic acid or vitamin C.

Of interest is the use of a nanodispersion wherein the non-aqueous polar organic solvents are polyols, homopolymeric or block copolymeric liquid ethers.

Preferably, step (α) is carried out in anhydrous medium; and step (β) is carried out without homogenisation.

Most preferably, the particles in the nanodispersion have an average diameter of <100 nm.

A preferred embodiment of this invention is the combination of the water-solube active ingredient, for example ascorbic acid, with another antioxidant [component (e)].

Preferably, component (e) is selected from the group consisting of carbon bridged hindered phenols, ester bridged hindered phenols, amide bridged hindered phenols, lactones of hindered phenoles, sterically hindered oxylamines and sterically hindered hydroxylamines.

Preferred antioxidants [component (e)] are compounds of formulae (1), (2) and/or (3)

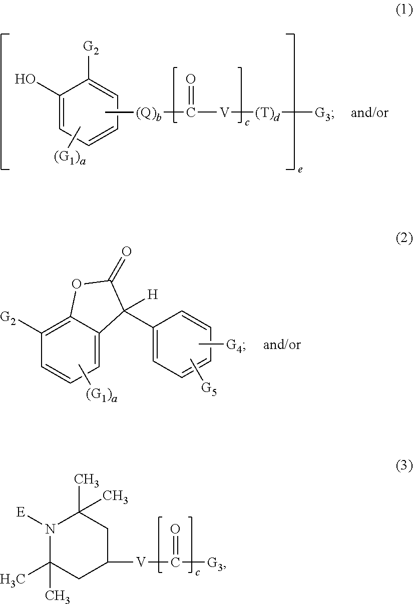

and acid addition salts thereof,
wherein in formulae (1), (2) and (3)
G₁ is hydrogen; C₁-C₂₂alkyl; C₁-C₂₂alkylthio; C₂-C₂₂alkylthioalkyl; C₅-C₇cycloalkyl; phenyl; C₇-C₉phenylalkyl; or SO₃M;
G₂ is C₁-C₂₂alkyl; C₅-C₇cycloalkyl; phenyl; or C₇-C₉phenylalkyl;
E is oxyl or hydroxyl;
Q is —CₘH₂ₘ—;

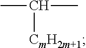

—CₘH₂ₘ—NH; a radical of formula

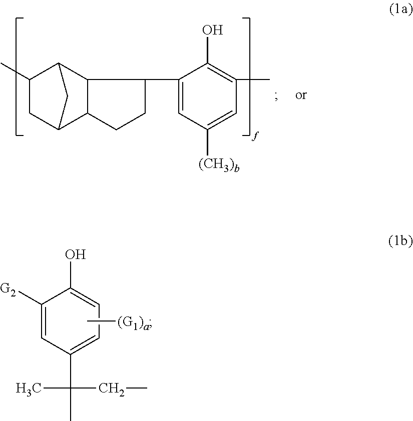

T is —CₙH₂ₙ—; —(CH₂)ₙ—O—CH₂—; phenylene;

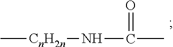

or a radical of
formula (1c)

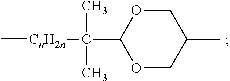

V is —O—; or —NH—;
a is 0; 1; or 2;
b, c and d and g are each independently of one another 0 or 1;
e is an integer from 1 to 4;
f is an integer from 1 to 3; and
m, n and p are each independently of one another an integer from 1 to 3;
q is 0 or an integer from 1 to 3;
if e=1, or in formula (3), then
G₃ is hydrogen; C₁-C₂₂alkyl; C₅-C₇cycloalkyl; C₁-C₂₂alkylthio; C₂-C₂₂alkylthioalkyl; C₂-C₁₈alkenyl; C₁-C₁₈phenylalkyl; M; SO₃M; a radical of formula

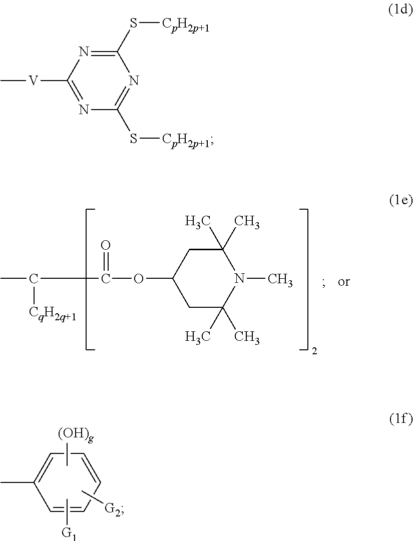

or G₃ is propyl substituted by OH and/or by C₂-C₂₂alkanoyloxy;
M is alkali; ammonium; H;
if e=2, then
G₃ is a direct bond; —CH₂—;

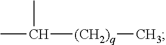

or —S—; or G₃ is propyl substituted by OH or C₂-C₂₂alkanoyloxy;
if
e=3, then
G₃ is the radical of formula

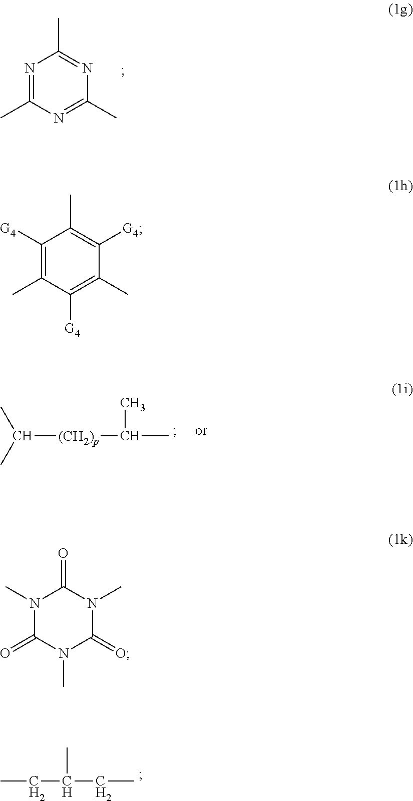

if
e=4, then
G₃ is

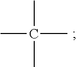

G₄ and G₅ are each independently of the other hydrogen; or C₁-C₂₂alkyl; and the compounds of the formulae (16), (18), (20), (21), (22), (23)

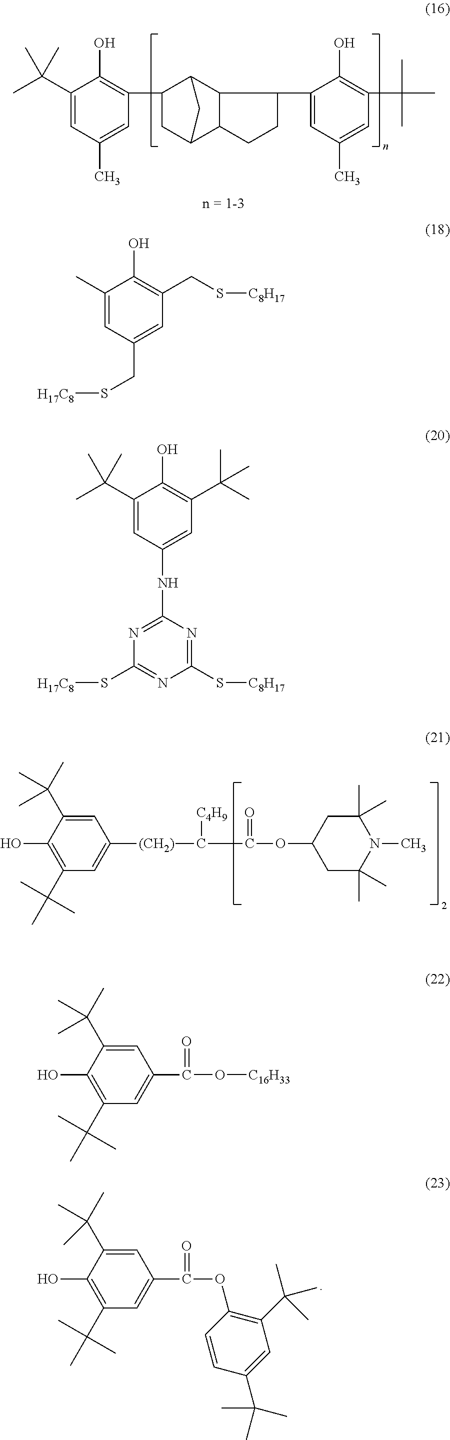

Most preferably, component (e) is a compound of the formula A or B

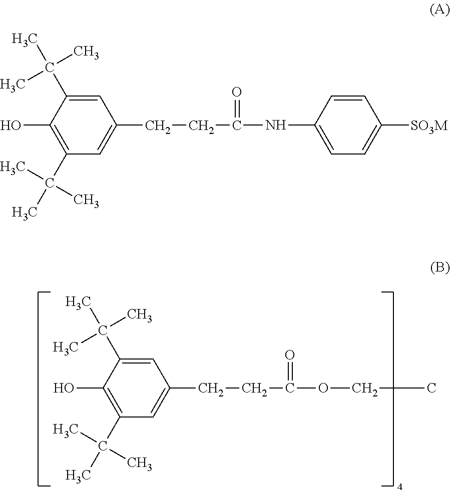

wherein M is hydrogen or sodium.

These two compounds of the formula A and B are commercially available by Ciba Specialty Chemicals Inc. Tinogard AO6® is the compound of the formula A. Tinogard TT® is the compound of the formula B.

Optionally, further hydrophilic or lipophilic antioxants can be used.

Examples of such further hydrophilic or lipophilic antioxidants are:
tocopherol (α, β, γ, δ isomers) and its esters of acids with general formulas

H(CH₂)ₙ(CHR)COOH (1)

CH₃(CH₂)ₘCH═CH(CH₂)ₙCOOH (2)
where R is hydrogen atom or OH group, m, n are integral numbers from 0 to 22 where m+n sum is maximally 22;
tocotrienol (α, β, γ, δ isomers), containing one unsaturated fatty chain, and its esters of acids;
ascorbic acid esters such as phosphoric acid and also sodium, potassium, lithium and magnesium salts, Ascorbyl Tetraisopalmitate, further ester with pyrrolidonecarboxylic acid and esters of acids with general formulas

H(CH₂)ₙ(CHR)COOH (3)

CH₃(CH₂)ₘCH═CH(CH₂)ₙCOOH (4)
where R is hydrogen atom or OH group, m, n are integral numbers from 0 to 20 where m+n sum is maximally 21;
retinoids include all natural and/or synthetic analogs of vitamin A or retinal-like compounds which possess the biological activity of vitamin A in the skin as well as the geometric isomers and stereoisomers of these compounds. Preferred compounds are retinal, retinol esters (e.g., C₂-C₂₂alkyl esters (saturated or unsaturated alkyl chains) of retinal, including retinyl palmitate, retinyl acetate, retinyl propionate, retinal, and/or retinoic acid (including all trans retinoic acid and/or 13-cis-retinoic acid) or derivatives; other retinoids which are useful herein are described for example in U.S. Pat. No. 4,677,120; U.S. Pat. No. 4,885,311; U.S. Pat. No. 5,049,584 or U.S. Pat. No. 5,124,356. Other suitable retinoids are tocopheryl-retinoate [tocopherol ester of retinoic acid (trans or cis)], adapalene [6-(3-(1-adamantyl)-4-methoxyphenyl)-2-naphtoic acid] and tazarotene (ethyl 6-[2-(4,4-dimethylthiochroman-6-yl)-ethynyl]nicotinate);
carotenoids such as α-, β-, γ-, and δ-carotene, lutein, xanthophylls, zeaxanthine, violaxanthine, cryptoxanthine, fukoxanthine, antheraxanthine, lycopene, didehydrolycopene and tetradehydrolycopene carotenoids;
enzymatic antioxidants such as glutathione peroxidase, catalase, superoxide dismutase;
ubiquinone and Idebenone(hydroxydecyl ubiquinone), ubiquinol and its derivatives;
lipoic acid and its derivatives such as alpha-lipoic acid;
rutinic acid and its derivatives such as α-glucosylrutin, a water soluble flavonoid, rutin hydrate (vitamin P);
botanical extracts such as white and green tea extracts, chicory leaf extract (cichorium intubybus), passionflower extract (passiflora incarnata), aspalathus linearis extract, rosmary extract, red leaf extract of aceraceae maple tree or of rosaceae cherry tree, curcuma longa L (curcuminoids active ingredients), leontopodium alpinum extract, emblica officinalis (phyllanthus emblica) tree extract;
phenolic acids such as caffeic acid, 3,4-dihydroxyphenyl acetic acid, 3,4-dihydroxybenzoic acid;
flavonoids and polyphenols such as flavanones selected from the group consisting of unsubstituted flavanones, mono-substituted flavanones, and mixtures thereof; chalcones selected from the group consisting of unsubstituted chalcones, mono-substituted chalcones, di-substituted chalcones, tri-substituted chalcones, and mixture thereof; flavones selected from the group consisting of unsubstituted flavones, mono-substituted flavones, di-substituted flavones, and mixtures thereof; or more isoflavones; coumarins selected from the group consisting of unsubstituted coumarins, mono-substituted coumarins, di-substituted coumarins, and mixtures thereof; flavonols, anthocyanins, catechins, proanthocyanidins (grape seed extract); flavonoids which are for examply broadly disclosed in U.S. Pat. No. 5,686,082 or U.S. Pat. No. 5,686,367 can also be used;
chlorogenic acid and ferulic acid.

Preferably, the further hydrophylic or lipophilic antioxidants are selected from the group consisting of tocopherols and its ester of acids, tocotrienol and its ester of acids, ascorbic acid esters, retinoids, carotenoids, glutathione peroxidise, catalase, superoxide dismutase, ubiquinone and idebenone, lipoic acid and its derivatives, rutinic acid and its derivatives, phenolic acids, flavonoids and polyphenols, chlorogenic acid and ferulic acid.

Preferably, the weight ratio between component (e) [further hydrophylic or lipophilic antioxidant] and component (d) [water-soluble ingredient such as for example ascorbic acid] is from 1:100 to 10:1.

In order for these substances to have an effect at the desired site, they must be transported to the respective site, for example to the skin surface, mucosae, nails, dental enamel or hair, but also to the epidermal and dermal areas of the skin, by means of so-called carrier and transport vehicles (carrier systems). For this purpose, many cosmetic active agents, for example water-soluble vitamins, the amino acid group or the water-soluble melanines, are encapsulated in liposomes. Owing to the physical and chemical properties of the membranes and the membrane-forming molecules, the use of said carrier systems in conventional cosmetic formulations is severely limited.

Conventionally structured membranes are highly susceptible to amphiphilic substances, such as ionogenic or non-ionogenic emulsifiers, fatty amines, amphoteric emulsifiers, detergent surfactants, thickeners or preservatives which form the basis for many cosmetic preparations or which are present as so-called active agents or excipients in cosmetic end formulations. The structure of the membranes changes in the presence of such substances, which may result in the destruction of the added carrier system during preparation.

Customary membranes are also exposed to the attack of acids and bases. Thus, it is required, for example, that carrier systems containing phospholipids can only be used in the range of the so-called pH optimum of about pH 6.5. Lowering or raising of the pH results in the hydrolysis of the membrane-forming molecules.

Surprisingly, it has now been found that nanodispersions of suitable composition can, in the presence of amphiphilic substances, be incorporated into cosmetic end formulations over a wide pH range in very simple manner while retaining their morphological and physicochemical properties.

An alternative process is to mix component (a) to the discontinuous phase which contains only a non aqueous polar organic solvent to fasten and improve the hydration rate of component (a). In this case, heating period of 20 to 30 minutes is required under optimum temperature between 70 and 80° C. and then cooling down to room temperature before adding the water-soluble active. Then, under moderate stirring, gradually the discontinuous phase to the continuous phase [components (b) and (c)] is added.

The nanodispersions characterised by the process contain particles having an average diameter of <300 nm, typically of less than 100 nm and most preferably from the range of 10 nm to 80 nm. The distribution is monodisperse and correspons to a Gaussian distribution.

A preferred use of a nanodispersion which comprises components (a), (b), (c) and (d) and optionally further components such as for example component (e) is wherein component (a) is a substance which is suitable for forming so-called bilayers, component (b) is a substance which preferably forms W/O structures, and component (c) is a functional lipophilic active agent customarily used in cosmetics.

The nanodispersion preferably contains as component (a) a phospholipid, a hydrated or partially hydrated phospholipid, a lysophospholipid, a ceramide, or mixtures of theseof.

A very particularly preferred phospholipid is that of the formula 1

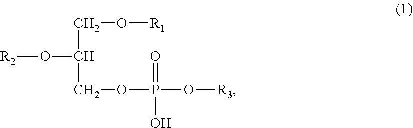

wherein
R₁ is C₁₀-C₂₀acyl;
R₂ is hydrogen or C₁₀-C₂₀acyl,
R₃ is hydrogen, 2-trimethylamino-1-ethyl (or referred as lecithin), 2-amino-1-ethyl; C₁-C₅alkyl which is unsubstituted or substituted by one or several carboxy, hydroxy or amino groups; the inositol or glyceryl group; or salts of these compounds of the formula 1.

C₁₀-C₂₀Acyl is preferably straight-chain C₁₀-C₂₀alkanoyl containing an even number of carbon atoms and straight-chain C₁₀-C₂₀alkenoyl containing a double bond and an even number of carbon atoms.

Straight-chain C₁₀-C₂₀alkanoyl containing an even number of carbon atoms is, for example, n-dodecanoyl, n-tetradecanoyl, n-hexadecanoyl or n-octadecanoyl.

Straight-chain C₁₀-C₂₀alkenoyl containing a double bond and an even number of carbon atoms is, for example, 6-cis- or 6-trans-, 9-cis- or 9-trans-dodecenoyl, -tetradecenoyl, -hexadecenoyl, -octadecenoyl or -eicosenoyl, preferably 9-cis-octa-decenoyl (oleoyl), and also 9,12-cis-octadecadienoyl or 9,12,15-cis-octadecatrienoyl.

A phospholipid of formula (I), wherein R₃ is 2-trimethylamino-1-ethyl, is referred to by the trivial name lecithin, and a phospholipid of formula (I), wherein R₃ is 2-amino-1-ethyl, by the trivial name cephalin. Suitable are, for example, naturally occurring cephalin or lecithin, e.g. cephalin or lecithin from soybeans or chicken eggs with different or identical acyl groups, or mixtures thereof.

The phospholipid of formula (1) may also be of synthetic origin. The expression “synthetic phospholipid” is used to define phospholipids having uniform composition with respect to R₁ and R₂. Such synthetic phospholipids are preferably the lecithins and cephalins defined above, wherein the acyl groups R₁ and R₂ have a defined structure and which are derived from a defined fatty acid having a degree of purity greater than about 95%. R₁ and R₂ may be identical or different and unsaturated or saturated. Preferably, R₁ is saturated, for example n-hexadecanoyl, and R₂ is unsaturated, for example 9-cis-octadecenoyl (oleoyl).

The expression “naturally occurring” phospholipid defines a phospholipid that does not have a uniform composition with respect to R₁ and R₂. Such natural phospholipids are likewise lecithins and cephalins, wherein the acyl groups R₁ and R₂ are derived from naturally occurring fatty acid mixtures.

The requirement “substantially pure” phospholipid of formula (1) defines a degree of purity of more than 90% by weight, preferably of more than 95% by weight of the phospholipid of formula (1), which can be demonstrated by means of suitable determination methods, for example by paper chromatography, thin-layer chromatography, by HPLC or by means of enzymatic colour testing.

In a phospholipid of formula (1), R₃ defined as C₁-C₄alkyl is, for example, methyl or ethyl. Methyl is preferred.

R₃ defined as C₁-C₅alkyl substituted by one or several carboxy, hydroxy or amino groups is, for example, 2-hydroxyethyl, 2,3-dihydroxy-n-propyl, carboxymethyl, 1- or 2-carboxyethyl, dicarboxymethyl, 2-carboxy-2-hydroxyethyl or 3-carboxy-2,3-dihydroxy-n-propyl, 3-amino-3-carboxy-n-propyl or 2-amino-2-carboxy-n-propyl, preferably 2-amino-2-carboxyethyl.

Phospholipids of formula (1) containing these groups can be present in salt form, for example as sodium or potassium salt.

Phospholipids of formula (1), wherein R₃ is the inositol or glyceryl group, are known by the names phosphatidylinositol and phosphatidylglycerol.

The acyl radicals in the phospholipids of formula (1) are also customarily known by the names given in brackets:

9-cis-dodecenoyl (lauroleoyl), 9-cis-tetradecenoyl (myristoleoyl), 9-cis-hexadecenoyl (palmitoleoyl), 6-cis-octadecenoyl (petroseloyl), 6-trans-octadecenoyl (petroselaidoyl), 9-cis-octadecenoyl (oleoyl), 9-trans-octadecenoyl (elaidoyl), 9,12-cis-octadecadienoyl (linoleoyl), 9,12,15-cis-octadecatrienoyl (linolenoyl), 11-cis-octadecenoyl (vaccenoyl), 9-cis-eicosenoyl (gadoleoyl), 5,8,11,14-cis-eicosatetraenoyl (arachidonoyl), n-dodecanoyl (lauroyl), n-tetradecanoyl (myristoyl), n-hexadecanoyl (palmitoyl), n-octadecanoyl (stearoyl), n-eicosanoyl (arachidoyl), n-docosanoyl (behenoyl), n-tetracosanoyl (lignoceroyl).

A salt of the phospholipid of formula (1) is preferably cosmetically acceptable. Salts are defined by the existence of salt-forming groups in the substituent R₃ and by the free hydroxyl group at the phosphorus atom. The formation of internal salts is also possible. Alkali metal salts, especially the sodium salt, are preferred.

In a particularly preferred embodiment of this invention, purified lecithin from soybeans of the quality LIPOID S 100 or S 75, or a lecithin defined in the monograph USP23/NF 18, is used.

Component (a) is preferably used in a concentration of about 0.1 to 30% by weight, based on the total weight of the components (a), (b), (c) and (d).

Component (b) is preferably an emulsifier or emulsifier mixtures forming the preferred W/O structures. The nanodispersion preferably contains as component (b) at least one emulsifier of the siloxane or organosiloxane type such as described for example in U.S. Pat. No. 5,411,744 or US-A-2001/0007653A1; or one emulsifier of the sorbitan derivative type such as non ethoxylated esters, for example sorbitan oleate, sorbitan laurate or sorbitan stearate, and ethoxylated ones, for example Polysorbate 20®, Polysorbate 40®, Polysorbate 60®, Polysorbate 80®, and/or very particularly traditional W/O emulsifier type such as for example described below, or a mixture of these substances.

Regarding siloxane or organosiloxane types, preferred emulsifiers are those represented as siloxane emulsifier/surfactant structures which can be W/O oriented types, liquid or solid at room temperature, and must be preferably non ionic ones having an HLB value between 2 to 6.

The polymeric organosiloxane emulsifier used in the invention may have any of the following generic formulas:

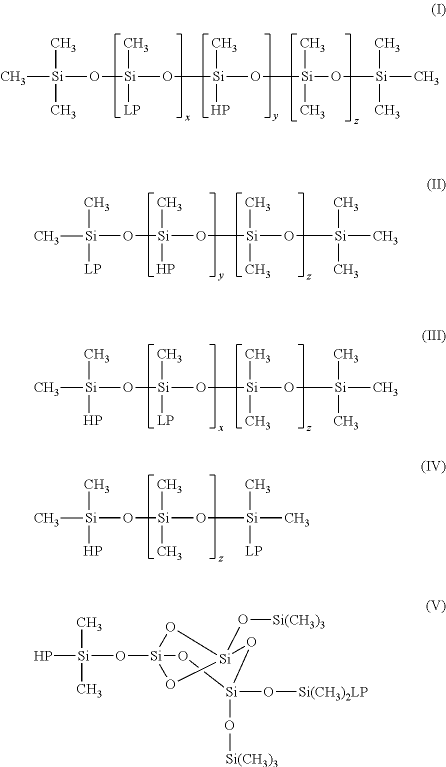

wherein LP is a lipophilic radical, HP is a hydrophilic radical,
x is 0 to 5000,
y is 0 to 5000, and
z is 0 to 5000, with the proviso that the organosiloxane contains at least one hydrophilic radical and at least one lipophilic radical.

Another type of preferred siloxane emulsifier suitable for use in the compositions of the invention as component (b) is a compound of the formula 2

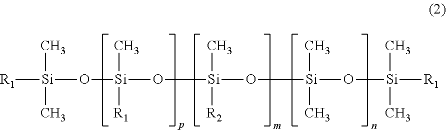

wherein
m is a number from 1 to 20,
n is a number from 0 to 500,
p is a number from 0 to 50,
R₁ is C₁-C₃₀alkyl or phenyl,
R₂ is —C_{c}C_{2c}—(OC₂H₄)ₐ—(OC₃H₆)_{b}—(OC₄H₈)_{d}—R₃,
R₃ is hydrogen, hydroxy, linear or branched C₁-C₁₂alkyl, linear or branched C₁-C₆alkoxy, linear or branched C₂-C₁₂acyloxy, —NHCH₂CH₂COOM, —NHCO(CH₂)_{d}—COOM, C₁-C₃₀-carboxyacyl, —NHCO(CH₂)_{d}—OH or —NH₃Y
M is hydrogen, sodium, potassium, lithium, ammonium or an organic ammonium,
Y is a monovalent organic or inorganic anion such as chloro, bromo, sulfate or carboxylate,
a is a number from 0 to 100,
b is a number from 0 to 50,
c is a number from 0 to 5, and
d is a number from 0 to 10.

Carboxylates are for example acetate, lactate or citrate.

These compounds represent the oxyalkylenated organo-modified silicones. Other denominations used are PEG/PPG dimethicones (dimethicone copolyols) or silicone polyethers that clearly show surface active properties necessary to emulsify.

Preferred silicone emulsifiers are compounds of the formula 3

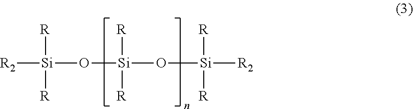

wherein
n is a number from 1 to 500,
R is linear or branched C₁-C₃₀alkyl radical or phenyl radical,
R₂ is —C_{c}H_{2c}—(OC₂H₄)ₐ—(OC₃H₆)_{b}—(OC₄H₈)_{d})_{d}—R₃, and
R₃, a, b, c and d have the same meaning as previously described.

A non exhaustive list of W/Si emulsifers is given in the following:

Oxyalkylenated organo-modified slicones:

PEG/PPG dimethicones & silicone polyethers
Bis-PEG/PPG-14/14 dimethicone
Bis-PEG/PPG-20/20 dimethicone
Bis-PEG/PPG-16/16 PEG/PPG-16/16-dimethicone
Bis-PEG-15 methyl ether dimethicone
Bis(PPG-7 undeceneth-21) dimethicone
Cetyl PEG/PPG-15/15 butyl ether dimethicone
Cetyl PEG/PPG-7/3 dimethicone
Cetyl PEG/PPG-10/1 dimethicone
Dimethicone copolyol
Dimethicone PEG-8 adipate
Dimethicone PEG-7 avocadoate
Dimethicone PEG-8 avocadoate
Dimethicone PEG-8 beeswax
Dimethicone PEG-n esters
Dimethicone/PEG-10 crosspolymer
Dimethicone/PEG-15 crosspolymer
Dimethicone/PEG-7 phosphate
Dimethicone/PEG-n phosphates
Dimethicone PEG/PPG-7/4 phosphate
Dimethicone PEG/PPG-12/4 phosphate
Dimethicone PEG-7 undecylenate
Laurylmethicone copolyol
PEG-10 dimethicone crosspolymer
PEG-12 dimethicone crosspolymer
PEG-10 lauryl dimethicone crosspolymer
PEG-15 lauryl dimethicone crosspolymer
PEG-6 methyl ether dimethicone
PEG-n methyl ether dimethicones
PEG-32 methyl ether dimethicone
PEG/PPG-20/22 butyl ether dimethicone
PEG/PPG-22/22 butyl ether dimethicone
PEG/PPG-23/23 butyl ether dimethicone
PEG/PPG-24/18 butyl ether dimethicone
PEG/PPG-27/9 butyl ether dimethicone
PEG/PPG-3/10 dimethicone
PEG/PPG-5/3 trisiloxane
PEG/PPG-n/m dimethicones
PEG/PPG-30/10 dimethicone
Potassium dimethicone PEG-7 phosphate
PPG-12 butyl ether dimethicone
PPG-12 dimethicone
PPG-27 dimethicone
TEA-dimethicone PEG-7 phosphate
Caprylyl dimethicone ethoxy glucoside
Dimethicone ethoxy glucoside
Dimethicone/polyglycerin-3 crosspolymer
PEG-9 polydimethylsiloxyethyl dimethicone
Polydimethylsiloxy PEG/PPG-24/19 butyl ether silsesquioxane
Polydimethylsiloxy PPG-13 butyl ether silsesquioxane
Polyglyceryl-3 disiloxane dimethicone
Polyglyceryl-3 polydimethylsiloxyethyl dimethicone
Sodium carboxydecyl PEG-8 dimethicone

Non-oxyalkylenated organo-modified silicones:

C₆-C₈Alkyl C₃-C₆alkyl glucoside dimethicone

Another type of preferred organosiloxane emulsifiers suitable for use in the compositions of the invention are those represented by the following generic formulas:

(CH₃Si)_{y-2}[(OSi(CH₃)₂)_{x/y}O-PE]_{y}

wherein PE is -(EO)ₘ(PO)ₙR,
R is lower alkyl or hydrogen,
EO is polyethyleneoxy,
PO is polypropyleneoxy,
m and n are each independently 1 to 5000, and
x and y are each independently 1 to 5000; and

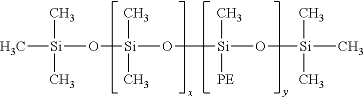

wherein PE=—CH₂CH₂CH₂O(EO)ₘ(PO)ₙZ,
Z is lower alkyl or hydrogen, and
m, n, x, y, EO and PO are as described above,
with the proviso that the molecule contains a lipophilic portion. Again, the lipophilic can be supplied by a sufficient number of methyl groups on the polymer backbone.

Particularly preferred is a Silwet® polymer of the following general formula:

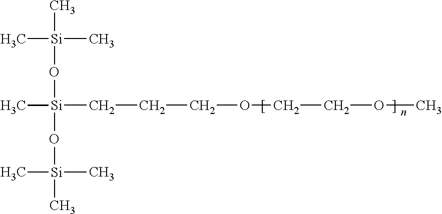

wherein n is 1-10, preferably 8.

Regarding traditional W/O emulsifier types, preferred emulsifiers are those represented as the following structure:

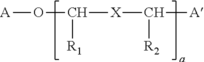

wherein a is 1 to 100,
R₁ and R₂ represent either hydrogen or methyl group —H or methyl
X represents the following group:

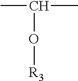

wherein R₃ is hydrogen, a branched or linear saturated or unsaturated alkyl or acyl group with 1 to 20 carbon atoms, and
A and A′ represent the same or a different organic group branched or linear saturated or unsaturated alkyl-, acyl- or hydroxyacyl radical with C₁₀ to C₃₀ as described in the formula I

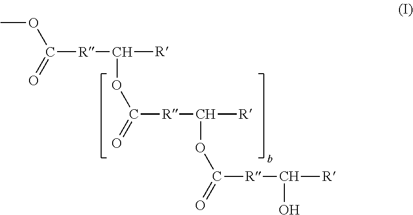

where R′ and R″ represent a branched or linear alkyl group with 1 to 20 carbon atoms, and b is a number from 0 to 200.

A, A′, R₁ and R₂ are most preferably hydrogen. Optionally the following groups can be selected: methyl, ethyl, propyl, isopropyl, myristoyl, palmitoyl, stearoyl, isostearoyl or eicosoyl. As alternatives the following structures can also be selected;

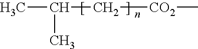

wherein n is a number from 10 to 20, or

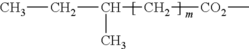

wherein m is a number from 9 to 19.

Preferred W/O systems are:
mono-, di-, tri- or tetra-glycerinesters or even of diesters of isostearic acid (such as polyglyceryl-n-diisostearate); for example polyglyceryl-4 isostearate, polyglyceryl-6 polyricinoleate, polyglyceryl-2 sesquiisostearate, distearoyl polyglyceryl-3 dimer dilinoleate, glyceryl 12-hydroxy-9-octodecenoate.
polyesters of hydroxystearic acid such as polyglyceryl-n-polyhydroxystearate or/and ethoxylated forms (such as PEG-30 dipolyhydroxystearate); for example polyglyceryl-10 pentahydroxystearate.
sorbitan mon- and di-esters of saturated and unsaturated fatty acids having from 6 to 22 carbon atoms and ethylene oxide products such as PEG-6 isostearate sorbitan, PEG-10 sorbitan laurate, sorbitan oleate, PEG-40 sorbitan peroleate.
ethoxylated oils or polymers/copolymers such as PEG-7 hydrogenated castor oil, PEG-45 dodecyl glycol copolymer.

Component (b), present in the nanodispersion according to this invention, may be used in an amount, for example, of 0.1 to 50% by weight, especially from 0.5 to 20% by weight and preferably from 1 to 10% by weight, based on the total weight of the components (a), (b), (c) and (d).

Component (c) is preferably a natural or synthetic or a partially synthetic di- or triglyceride, a mineral oil, silicone oil, wax, fatty alcohol, guerbet alcohol or the ester thereof, or a mixture of these substances.

The main categories of lipophilic emollients are represented as follows:

Esters which include only esters not otherwise classified as alkoxylated carboxylic acids, glyceryl esters, isethionates, lanolin derivatives, phosphorus compounds, sulfosuccinates or sulfuric acid esters.

Chemically, esters are the covalent compounds formed between acids and alcohols. Esters can be formed from almost all acids (inorganic and carboxylic) and any alcohol.

Thus, the number of naturally occurring and synthetically prepared esters used in cosmetics is very large. In order to subdivide the list of esters, most have been assigned to other chemical classes on the basis of some additional chemical features: alkoxylated fatty acids, alkyl ether sulfates, alkyl sulfates, fats and oils, glyceryl esters, phosphorus compounds, isothionates, sorbitan derivatives, sulfosuccinates, sulfuric acid derivatives and waxes.

The esters that belong to this specific class are derived from carboxylic acids and have the class RCO—OR′ where RCO— represents the carboxylic acid radical and where —OR′ is the alcohol residue. Included are low and medium molecular weight compounds, e.g., butyl acetate or isopropyl myristate; some polymers, e.g., polyvinyl acetate. They are subject to hydrolysis into alcohols and acids at extreme pHs and at elevated temperatures.

Esters in this group find diverse uses in cosmetics as emollients and conditioners (isopropyl myristate), as solvents (ethyl acetate), as fragrance components (methyl salicylate), and as preservatives (propylparaben).

Acetyl Triethylhexyl Citrate, Acetyl Trihexyl Citrate, Acetylated Hydrogenated Castor Oil, Acetylated Sucrose Distearate, Arachidyl Erucate, Arachidyl Propionate, Ascorbyl Tetraisopalmitate, Ascorbyl Tocopheryl Maleate, Behenyl Benzoate, Benzyl Laurate, Benzyl Ursolate, Bis(Trioctyldodecyl Citryl) Dilinoleate, Bis-Behenyl/Isostearyl/Phytosteryl Dimer Dilinoleyl Dimer Dilinoleate, Bis-Behenyl/Phytosteryl Dimer Dilinoleate, Bis-Isostearyl Dimer Dilinoleyl Dimer Dilinoleate, Butoxyethyl Stearate, Butyl Acetyl Ricinoleate, Butyl Babassuate, Butyl Isostearate, Butyl Myristate, Butyl Oleate, Butyl Stearate, Butylene Glycol Behenate, Butylene Glycol Laurate, Butylene Glycol Myristate, Butylene Glycol Oleate, Butylene Glycol Palmitate, Butylene Glycol Stearate, Butyloctyl Benzoate, C10-40 Isoalkyl Acid Phytosterol Esters, C11-15 Pareth-12 Stearate, C11-15 Pareth-3 Oleate, C11-15 Pareth-3 Stearate, C12-13 Alkyl Ethylhexanoate, C12-13 Alkyl Lactate, C12-15 Alkyl Benzoate, C12-15 Alkyl Ethylhexanoate, C12-15 Alkyl Lactate, C12-15 Pareth-12 Oleate, C12-15 Pareth-3 Benzoate, C12-15 Pareth-9 Hydrogenated Tallowate, C14-15 Dialkyl Carbonate, C14-16 Glycol Palmitate, C14-18 Acid Glycol Ester, C14-18 Alkyl Ethylhexanoate, C16-17 Alkyl Benzoate, C18-20 Glycol Isostearate, C18-28 Alkyl Acetate, C18-30 Acid Glycol Ester, C18-36 Acid Glycol Ester, C20-30 Glycol Isostearate, C23-43 Acid Pentaerythritol Tetraester, C32-36 Isoalkyl Stearate, Castor Oil Benzoate, Ceteareth-6 Olivate, Cetearyl Ethylhexanoate, Cetearyl Isononanoate, Cetearyl Palmitate, Cetyl Acetate, Cetyl Babassuate, Cetyl C12-15 Pareth-8 Carboxylate, Cetyl C12-15-Pareth-9 Carboxylate, Cetyl Caprate, Cetyl Caprylate, Cetyl Dimethyloctanoate, Cetyl Ethylhexanoate, Cetyl Glycol Isostearate, Cetyl Isononanoate, Cetyl Kombo Butterate, Cetyl Lactate, Cetyl Laurate, Cetyl Oleate, Cetyl PPG-2 Isodeceth-7 Carboxylate, Chimyl Isostearate, Chimyl Stearate, Cholesteryl Succinate, Coco-Caprylate/Caprate, Coco-Rapeseedate, Cycloartenyl Ferulate, Decyl Castorate, Decyl Hempseedate, Decyl Isostearate, Decyl Jojobate, Decyl Oleate, Decyl Palmitate, Decyl Succinate, Dibutyl Adipate, Dibutyl Sebacate, Dibutyldecyl IPDI, Dibutyloctyl IPDI, Dibutyloctyl Malate, Dibutyloctyl Sebacate, Di-C12-13 Alkyl Malate, Di-C12-13 Alkyl Tartrate, Di-C12-15 Alkyl Adipate, Di-C12-15 Alkyl Fumarate, Di-C12-15 Alkyl Maleate, Di-C14-15 Alkyl Tartrate, Dicaprylyl Carbonate, Dicaprylyl Maleate, Dicetyl Adipate, Dicocoyl Pentaerythrityl Distearyl Citrate, Diethyl Adipate, Diethyl Palmitoyl Aspartate, Diethyl Sebacate, Diethylene Glycol Diethylhexanoate/Diisononanoate, Diethylene Glycol Diisononanoate, Diethylhexyl 2,6-Naphthalate, Diethylhexyl Adipate, Diethylhexyl Carbonate, Diethylhexyl Fumarate, Diethylhexyl Malate, Diethylhexyl Maleate, Diethylhexyl Succinate, Diglyceryl Sebacate/Isopalmitate, Diglyceryl Stearate Malate, Diheptylundecyl Adipate, Dihexyl Adipate, Dihexyldecyl Adipate, Dihexyldecyl IPDI, Dihexyldecyl Lauroyl Glutamate, Dihexyldecyl Sebacate, Dihydrocholesteryl Butyrate, Dihydrocholesteryl Isostearate, Dihydrocholesteryl Macadamiate, Dihydrocholesteryl Nonanoate, Dihydrocholesteryl Octyldecanoate, Dihydrocholesteryl Oleate, Diisoamyl Malate, Diisobutyl Adipate, Diisocetyl Adipate, Diisocetyl Dodecanedioate, Diisodecyl Adipate, Diisononyl Adipate, Diisooctyl Adipate, Diisopropyl Adipate, Diisopropyl Dimer Dilinoleate, Diisopropyl Sebacate, Diisostearyl Adipate, Diisostearyl Fumarate, Diisostearyl Glutarate, Diisostearyl Malate, Diisostearyl Sebacate, Dilauryl Citrate, Dimer Dilinoleyl Diisostearate, Dimer Dilinoleyl Dimer Dilinoleate, Dimer Dilinoleyl Isostearate, Dimethicone PEG-7 Avocadoate, Dimethicone PEG-7 Cocoate, Dimethicone PEG-7 Isostearate, Dimethicone PEG-7 Octyldodecyl Citrate, Dimethicone PEG-7 Olivate, Dimethicone PEG-7 Phthalate, Dimethicone PEG-8 Adipate, Dimethicone PEG-8 Beeswax, Dimethicone PEG-8 Borageate, Dimethicone PEG-8 Meadowfoamate, Dimethiconol Borageate, Dimethiconol Dhupa Butterate, Dimethiconol Illipe Butterate, Dimethiconol Kokum Butterate, Dimethiconol Lactate, Dimethiconol Meadowfoamate, Dimethiconol Mohwa Butterate, Dimethiconol Sal Butterate, Dimethyl Adipate, Dimethyl Succinate, Dimyristyl Tartrate, Dioctadecanyl Didecyltetradecanoate, Dioctadecanyl Ditetradecyloctadecanoate, Dioctyldodeceth-2 Lauroyl Glutamate, Dioctyldodeceth-5 Lauroyl Glutamate, Dioctyldodecyl Adipate, Dioctyldodecyl Lauroyl Glutamate, Dioctyldodecyl Sebacate, Dipentaerythrityl Hexahydroxystearate/Hexastearate/Hexahydrogenated Rosinate, Dipentaerythrityl Pentaisononanoate, Dipentaerythrityl Pentaisostearate, Di-PPG-2 Myreth-10 Adipate, Di-PPG-3 Ceteth-4 Adipate, Di-PPG-3 Myristyl Ether Adipate, Dipropyl Adipate, Dipropylene Glycol Caprylate, Dipropylene Glycol Dibenzoate, Dipropylene Glycol Salicylate, Disteareth-2 Lauroyl Glutamate, Disteareth-5 Lauroyl Glutamate, Distearyl Citrate, Ditridecyl Adipate, Ditrimethylolpropane Isostearate/Sebacate, Ditrimethylolpropane Myristate/Stearate, Ditrimethylolpropane Triethylhexanoate, Erythrityl Triethylhexanoate, Ethoxydiglycol Behenate, Ethoxydiglycol Isostearate, Ethoxydiglycol Oleate, Ethyl Almondate, Ethyl Apricot Kernelate, Ethyl Arachidonate, Ethyl Avocadate, Ethyl Caprate, Ethyl Isostearate, Ethyl Jojobate, Ethyl Laurate, Ethyl Linoleate, Ethyl Linolenate, Ethyl Macadamiate, Ethyl Minkate, Ethyl Morrhuate, Ethyl Myristate, Ethyl Oleate, Ethyl Olivate, Ethyl Palmitate, Ethyl Pelargonate, Ethyl Persate, Ethyl Ricinoleate, Ethyl Stearate, Ethyl Turtlate, Ethyl Undecylenate, Ethyl Wheat Germate, Ethylhexyl Acetoxystearate, Ethylhexyl Adipate/Palmitate/Steparate, Ethylhexyl Benzoate, Ethylhexyl Benzyldioxopiperazyl Acetate, Ethylhexyl Caprylate/-Caprate, Ethylhexyl Cocoate, Ethylhexyl Ethylhexanoate, Ethylhexyl Hydroxystearate, Ethyl-hexyl Hydroxystearate Benzoate, Ethylhexyl Hydroxystearoyl Hydroxystearate, Ethylhexyl Isononanoate, Ethylhexyl Isopalmitate, Ethylhexyl Isostearate, Ethylhexyl Laurate, Ethylhexyl Linoleoyl Stearate, Ethylhexyl Myristate, Ethylhexyl Neopentanoate, Ethylhexyl Oleate, Ethylhexyl Palmitate, Ethylhexyl Pelargonate, Ethylhexyl Stearate, Glycereth-5 Lactate, Glycereth-7 Benzoate, Glycereth-7 Diisononanoate, Glycereth-7 Lactate, Glyceryl Stearate Dimer Dilinoleate Copolymer, Glyceryl Triacetyl Hydroxystearate, Glyceryl Triacetyl Ricinoleate, Glycol Cetearate, Glycol Dipalmate/Palm Kernelate/Olivate/Macadamiate, Glycol Dipivalate, Glycol Ethylhexanoate, Glycol Hydroxystearate, Glycol Montanate, Glycol Oleate, Glycol Palmitate, Glycol Ricinoleate, Glycol Stearate, Glycol Tallate, Glycol/Butylene Glycol Montanate, Heptylundecyl Hydroxystearate, Hexanediol Beeswax, Hexanediol Disalicylate, Hexanetriol Beeswax, Hexyl Isostearate, Hexyl Laurate, Hexyldecyl Benzoate, Hexyldecyl Ethyl hexanoate, Hexyldecyl Hexyldecanoate, Hexyldecyl Laurate, Hexyldecyl Myristoyl Methylaminopropionate, Hexyldecyl Stearate, Hydrogenated Ethylhexyl Olivate, Hydrogenated Ethylhexyl Sesamate, Hydroxycetyl Isostearate, Hydroxypropyl Dimethicone Behenate, Isoamyl Laurate, Isobutyl Myristate, Isobutyl Palmitate, Isobutyl Pelargonate, Isobutyl Stearate, Isobutyl Tallowate, Isocetyl Ethyl hexanoate, Isocetyl Isodecanoate, Isocetyl Isostearate, Isocetyl Laurate, Isocetyl Linoleoyl Stearate, Isocetyl Palmitate, Isocetyl Stearate, Isodeceth-2 Cocoate, Isodecyl Citrate, Isodecyl Cocoate, Isodecyl Ethylhexanoate, Isodecyl Hydroxystearate, Isodecyl Isononanoate, Isodecyl Laurate, Isodecyl Myristate. Isodecyl Neopentanoate, Isodecyl Oleate, Isodecyl Palmitate, Isodecyl Stearate, Isohexyl Caprate, Isohexyl Laurate, Isohexyl Neopentanoate, Isohexyl Palmitate, Isomerized Jojoba Oil, Isononyl Ferulate, Isononyl Isononanoate, Isopropyl Arachidate, Isopropyl Avocadate, Isopropyl Babassuate, Isopropyl Behenate, Isopropyl C12-15-Pareth-9 Carboxylate, Isopropyl Hydroxystearate, Isopropyl Isostearate, Isopropyl Jojobate, Isopropyl Laurate, Isopropyl Linoleate, Isopropyl Myristate, Isopropyl Oleate, Isopropyl Palmitate, Isopropyl PPG-2 Isodeceth-7 Carboxylate, Isopropyl Ricinoleate, Isopropyl Stearate, Isopropyl Tallowate, Isosorbide Dicaprylate, Isosorbide Laurate, Isosteareth-10 Stearate, Isostearyl Acetate, Isostearyl Acetyl Glutaminate, Isostearyl Avocadate, Isostearyl Benzoate, Isostearyl Ethylhexanoate, Isostearyl Ferulate. Isostearyl Glycolate, Isostearyl Hydroxystearate, Isostearyl Isononanoate, Isostearyl Isostearate, Isostearyl Lactate, Isostearyl Laurate, Isostearyl Linoleate, Isostearyl Myristate, Isostearyl Neopentanoate, Isostearyl Palmitate, Isotridecyl Isononanoate, Isotridecyl Stearate, Kojic Dipalmitate, Kojic Isopalmitate, Laneth-10 Acetate, Laneth-9 Acetate, Laureth-2 Acetate, Laureth-2 Benzoate, Laureth-2 Ethyl hexanoate, Lauryl Cocoate, Lauryl Ethyl hexanoate, Lauryl Isostearate, Lauryl Lactate, Laurylgluconamide Palmitates, Lignoceryl Erucate, Linoleyl Lactate, Meadowfoam Estolide, Methyl Acetyl Ricinoleate, Methyl Behenate, Methyl Benzoate, Methyl Caproate, Methyl Caprylate, Methyl Caprylate/Caprate, Methyl Cocoate, Methyl Dehydroabietate, Methyl Gluceth-20 Benzoate, Methyl Glucose Caprylate/Caprate, Methyl Glucose Dioleate, Methyl Glucose Isostearate, Methyl Glucose Laurate, Methyl Glucose Sesquicaprylate/Sesquicaprate, Methyl Glucose Sesquicocoate, Methyl Glucose Sesquiisostearate, Methyl Glucose Sesquilaurate, Methyl Glucose Sesquioleate, Methyl Glucose Sesquistearate, Methyl Hydrogenated Rosinate, Methyl Hydroxystearate, Methyl Isostearate, Methyl Laurate, Methyl Linoleate, Methyl Myristate, Methyl Oleate, Methyl Palmate, Methyl Palmitate, Methyl Pelargonate, Methyl Ricinoleate, Methyl Rosinate, Methyl Soyate, Methyl Stearate, Methyl Su nflowerseedate, Monoglycerides Acetate, Monoglycerides Citrate, Monoglycerides Lactate, Monoglycerides Succinate, Monoglycerides/Diacetyl Tartarate Esters, Myreth-2 Myristate, Myreth-3 Caprate, Myreth-3 Ethylhexanoate, Myreth-3 Laurate, Myreth-3 Myristate, Myreth-3 Palmitate, Myristyl Acetate, Myristyl Alaninate, Myristyl Ethylhexanoate, Myristyl Isostearate, Myristyl Lactate, Myristyl Lignocerate, Myristyl Neopentanoate, Myristyl Propionate, Neopentyl Glycol Dicaprate, Neopentyl Glycol Dicaprylate/Dicaprate, Neopentyl Glycol Dicaprylate/Dipelargonate/Dicaprate, Neopentyl Glycol Diethylhexanoate, Neopentyl Glycol Diheptanoate, Neopentyl Glycol Dilaurate, Nonyl Acetate, Octyldodecyl Beeswax, Octyldodecyl Benzoate, Octyldodecyl Ethylhexanoate, Octyldodecyl Lactate, Octyldodecyl Neodecanoate, Octyldodecyl Neopentanoate, Octyldodecyl PCA, Octyldodecyl/PPG-3 Myristyl Ether Dimer Dilinoleate, Oleth-2 Benzoate, Oleuropein Aglycone, Oleyl Acetate, Oleyl Lactate, Oleyl Oleate, Palmitoyl Inulin, PEG-20 Methyl Glucose Distearate, PEG-20 Methyl Glucose Sesquicaprylate/Sesquicaprate, PEG-20 Methyl Glucose Sesquilaurate, PEG-3 Butylene Glycol Laurate, PEG-3 Trimethylolpropane Tristearate, PEG-4 Trimethylolpropane Distearate, PEG-5 Tricapryl Citrate, PEG-5 Tricetyl Citrate, PEG-5 Trilauryl Citrate, PEG-5 Trimethylolpropane Trimyristate, PEG-5 Trimyristyl Citrate, PEG-5 Tristearyl Citrate, PEG-6 Butylene Glycol Laurate, PEG-6 Propylene Glycol Caprylate/Caprate, PEG-8 Propylene Glycol Cocoate, PEG-80 Methyl Glucose Laurate, Pentaerythrityl Dioleate, Pentaerythrityl Distearate, Pentaerythrityl Rosinate, Pentaerythrityl Stearate, Pentaerythrityl Tetrabehenate/Benzoate/Ethylhexanoate, Pentaerythrityl Tetrabenzoate, Pentaerythrityl Tetraethylhexanoate/Benzoate, Pentaerythrityl Tetraethylhexanoate/Tetramethoxycinnamate, Pentaerythrityl Trioleate, Phenethyl Benzoate, Phytosteryl Hydroxystearate, Phytosteryl Isostearyl Dimer Dilinoleate, Phytosteryl Rice Branate, Poloxamer 182 Dibenzoate, Polyperfluoroethoxymethoxy Difluoroethyl PEG Diisostearate, PPG-15 Stearyl Ether Benzoate, PPG-2 lsoceteth-20 Acetate, PPG-20 Methyl Glucose Ether Distearate, PPG-3 Dipivalate, PPG-3 Myristyl Ether Neoheptanoate, Propyl C12-15 Pareth-8 Carboxylate, Propylene Glycol Behenate, Propylene Glycol Caprylate, Propylene Glycol Ceteth-3 Acetate, Propylene Glycol Ceteth-3 Propionate, Propylene Glycol Cocoate, Propylene Glycol Dibenzoate, Propylene Glycol Heptanoate, Propylene Glycol Hydroxystearate, Propylene Glycol Isoceteth-3 Acetate, Propylene Glycol Isostearate, Propylene Glycol Laurate, Propylene Glycol Linoleate, Propylene Glycol Linolenate, Propylene Glycol Myristate, Propylene Glycol Myristyl Ether Acetate, Propylene Glycol Oleate, Propylene Glycol Ricinoleate, Propylene Glycol Soyate, Propylene Glycol Stearate, Quercetin Caprylate, Raffinose Myristate, Raffinose Oleate, Retinyl Formyl Aspartamate, Salicylyl Beeswax, Starch Tallowate, Steareth-9 Stearate, Stearyl Acetate, Stearyl Acetyl Glutaminate, Stearyl Benzoate, Stearyl Citrate, Stearyl Glycol Isostearate, Stearyl Lactate, Stearyl/PPG-3 Myristyl Ether Dimer Dilinoleate, Sucrose Acetate/Stearate, Sucrose Cocoate, Sucrose Dilaurate, Sucrose Distearate, Sucrose Hexaerucate, Sucrose Laurate, Sucrose Mortierellate, Sucrose Myristate. Sucrose Oleate, Sucrose Palmitate, Sucrose Pentaerucate, Sucrose Polybehenate, Sucrose Polycottonseedate, Sucrose Polylaurate, Sucrose Polylinoleate, Sucrose Polyoleate, Sucrose Polypalmate, Sucrose Polysoyate, Sucrose Polystearate, Sucrose Ricinoleate, Sucrose Stearate, Sucrose Tetraisostearate, Sucrose Tetrastearate Triacetate, Sucrose Tribehenate, Sucrose Tristearate, Tetradecylpropionates, Tetrahydrofurfuryl Ricinoleate, Tocopheryl Retinoate, Tribenzoyl Triricinolein, Tri-C12-13 Alkyl Citrate, Tri-C14-15 Alkyl Citrate, Tricaprylyl/Capryl Trimellitate, Tridecyl Ethylhexanoate, Tridecyl Isononanoate, Tridecyl Neopentanoate, Tridecyl Stearate, Triethylhexyl Citrate, Triethylhexyl Trimellitate, Triisodecyl Trimellitate, Triisopropyl Citrate, Trioleyl Citrate.

Glyceryl esters and derivatives (excluding fats and oils) such as glyceryl esters comprise a subgroup of esters that are primarily fatty acid mono- and diglycerides or triglycerides modified by reaction with other alcohols and the like. Also included are the fatty acid esters of polyglycerin, the complex dehydration product of glycerin, e.g., polyglyceryl-4 Isostearate.

Like natural fats and oils, these glyceryl esters are subject to hydrolysis. They are used in cosmetics as emollients, lubricants, vehicles, and thickening agents. In addition, the mono-glycerides (e.g., glyceryl stearate) are important emulsifiers. Examples are:

Acetyl Glyceryl Ricinoleate, Acetylated Lard Glyceride, Acetylated Palm Kernel Glycerides, Argan Oil PEG-8 Esters, Bis-Diglyceryl Polyacyladipate-1, Bis-Diglyceryl Polyacyladipate-2, C10-40 Isoalkyl Acid Glyceride, C10-40 Isoalkyl Acid Triglyceride, Canola Oil Glyceride, Caprylic/Capric Glycerides, Caprylic/Capric/Coco Glycerides, Caprylic/Capric/Succinic Triglyceride, Cocoglycerides Cottonseed Glyceride, DATEM, Di-Glyceryl Stearate Dimer Dilinoeate, Diglyceryl Stearate Malate, Diisostearoyl Polyglyceryl-3 Dimer Dilinoleate, Ethylhexyloxyglyceryl Palmitate, Fish Glycerides, Glycereth-18 Benzoate, Glycereth-20 Benzoate, Glycereth-6 C10-18 Acidate, Glycereth-7 Triacetate, Glycerin/Phthalic Acid Copolymer Hydroxypropyl C9-11 Isoalkyl Acidate, Glyceryl Abietate, Glyceryl Abietate/Maleate, Glyceryl Adipate, Glyceryl Alginate, Glyceryl Arachidate, Glyceryl Arachidonate, Glyceryl Behenate, Glyceryl Behenate/Eicosadioate, Glyceryl Caprate, Glyceryl Caprylate, Glyceryl Caprylate/Caprate, Glyceryl Citrate/Lactate/Linoleate/Oleate, Glyceryl Cocoate, Glyceryl Cocoate/Citrate/Lactate, Glyceryl Collagenate, Glyceryl Diarachidate, Glyceryl Dibehenate, Glyceryl Dibehenate/Hydrogenated Rosinate, Glyceryl Dierucate, Glyceryl Di-Hydroxystearate, Glyceryl Diisopalmitate, Glyceryl Diisostearate, Glyceryl Dilaurate, Glyceryl Dilinoleate, Glyceryl Dimyristate, Glyceryl Dimyristate/Hydrogenated Rosinate, Glyceryl Dioleate, Glyceryl Dipalmitate, Glyceryl Dipalmitoleate, Glyceryl Diricinoleate, Glyceryl Distearate, Glyceryl Erucate, Glyceryl Heptanoate, Glyceryl Hydrogenated Rosinate, Glyceryl Hydrogenated Soyate, Glyceryl Hydroxystearate, Glyceryl Isopalmitate, Glyceryl Isostearate, Glyceryl Isostearate/Myristate, Glyceryl Isostearates, Glyceryl Isotridecanoate/Stearate/Adipate, Glyceryl Lanolate, Glyceryl Laurate, Glyceryl Laurate/Oleate, Glyceryl Linoleate, Glyceryl Linolenate, Glyceryl Montanate, Glyceryl Myristate, Glyceryl Oleate, Glyceryl Oleate/Elaidate, Glyceryl Olivate, Glyceryl Palmitate, Glyceryl Palmitate Lactate, Glyceryl Palmitate/Stearate, Glyceryl Palmitoleate, Glyceryl Ricinoleate, Glyceryl Ricinoleate SE, Glyceryl Rosinate, Glyceryl Sesquioleate, Glyceryl Stearate, Glyceryl Stearate Citrate, Glyceryl Stearate Lactate, Glyceryl Stearate Succinate, Glyceryl Stearate/Acetate, Glyceryl Stearate/Malate, Glyceryl Stearates, Glyceryl Tallowate, Glyceryl Tripalmate/Palm Kernelate/Olivate/Macadamiate/Rapeseedate, Glyceryl Undecylenate, Glyceryl/Sorbitol Oleate/Hydroxystearate, Hydrogenated Castor Oil/Sebacic Acid Copolymer, Hydrogenated Coco-Glycerides, Hydrogenated Lard Glyceride, Hydrogenated Lard Glycerides, Hydrogenated Palm Glycerides, Hydrogenated Palm Glycerides Citrate, Hydrogenated Palm Kernel Glycerides, Hydrogenated Soy Glycerides, Hydrogenated Tallow Glyceride Citrate, Hydrogenated Tallow Glyceride Lactate, Hydrogenated Tallow Glycerides, Hydrogenated Tallow Glycerides Citrate, Hydrogenated Vegetable Glycerides, Hydrogenated Vegetable Glycerides Citrate, Hydroxylated Milk Glycerides, Hydroxystearic/Linolenic/Oleic Polyglycerides, Isostearic/Myristic Glycerides, Lard Glyceride, Lard Glycerides, Lauryl Polyglyceryl-6 Cetearyl Glycol Ether, Methylsilanol PEG-7 Glyceryl Cocoate, Methylsilanol Tri-PEG-8 Glyceryl Cocoate, Oleic/Linoleic/Linolenic Polyglycerides, Palm Glycerides, Palm Kernel Glycerides, PEG-10 Castor Oil, PEG-10 Glyceryl Triisostearate, PEG-10 Glyceryl Trioleate, PEG-10 Glyceryl Tristearate, PEG-10 Hydrogenated Castor Oil, PEG-10 Olive Glycerides, PEG-10 Sunflower Glycerides, PEG-11 Avocado Glycerides, PEG-11 Babassu Glycerides, PEG-11 Castor Oil, PEG-11 Cocoa Butter Glycerides, PEG-12 Glyceryl Dioleate, PEG-12 Glyceryl Distearate, PEG-12 Palm Kernel Glycerides, PEG-13 Mink Glycerides, PEG-13 Sunflower Glycerides, PEG-14 Avocado Glycerides, PEG-15 Castor Oil, PEG-15 Glyceryl Laurate, PEG-15 Glyceryl Triisostearate, PEG-15 Glyceryl Trioleate, PEG-16 Castor Oil, PEG-16 Hydrogenated Castor Oil, PEG-16 Macadamia Glycerides, PEG-18 Glyceryl Oleate/Cocoate, PEG-18 Palm Glycerides, PEG-192 Apricot Kernel Glycerides, PEG-2 Castor Oil, PEG-2 Hydrogenated Castor Oil, PEG-2 Olive Glycerides, PEG-2 Sunflower Glycerides, PEG-20 Almond Glycerides, PEG-20 Corn Glycerides, PEG-20 Evening Primrose Glycerides, PEG-20 Glyceryl Stearate, PEG-20 Glyceryl Triisostearate, PEG-20 Glyceryl Tristearate, PEG-20 Hydrogenated Palm Glycerides, PEG-25 Glyceryl Trioleate, PEG-25 Moring a Glycerides, PEG-3 Castor Oil, PEG-3 Glyceryl Cocoate, PEG-3 Glyceryl Triisostearate, PEG-3 Glyceryl Trioleate, PEG-3 Glyceryl Tristearate, PEG-30 Glyceryl Triisostearate, PEG-35 Almond Glycerides, PEG-4 Caprylic/Capric Glycerides, PEG-4 Castor Oil, PEG-4 Glyceryl Distearate, PEG-4 Glyceryl Tristearate, PEG-40 Glyceryl Triisostearate, PEG-40 Olive Glycerides, PEG-42 Babassu Glycerides, PEG-42 Mushroom Glycerides, PEG-45 Palm Kernel Glycerides, PEG-45 Safflower Glycerides, PEG-5 Castor Oil, PEG-5 Glyceryl Triisostearate, PEG-5 Glyceryl Trioleate, PEG-5 Glyceryl Tristearate, PEG-5 Hydrogenated Castor Oil, PEG-5 Hydrogenated Corn Glycerides, PEG-50 Glyceryl Triisostearate, PEG-6 Almond Glycerides, PEG-6 Caprylic/Capric Glycerides, PEG-6 Glyceryl Caprate, PEG-6 Glyceryl Tristearate, PEG-6 Hydrogenated Castor Oil, PEG-6 Hydrogenated Palm/Palm Kernel Glyceride, PEG-6 Olive Glycerides, PEG-60 Almond Glycerides, PEG-60 Corn Glycerides, PEG-60 Glyceryl Triisostearate, PEG-60 Shea Butter Glycerides, PEG-65 Hydrogenated Castor Oil, PEG-7 Caprylic/Capric Glycerides, PEG-7 Glyceryl Cocoate, PEG-7 Glyceryl Laurate, PEG-7 Glyceryl Soyate, PEG-7 Hydrogenated Castor Oil, PEG-7 Olive Glycerides, PEG-7 Sunflower Glycerides, PEG-70 Mango Glycerides, PEG-75 Cocoa Butter Glycerides, PEG-75 Meadowfoam Oil, PEG-75 Shea Butter Glycerides, PEG-75 Shorea Butter Glycerides, PEG-75 Soy Glycerides, PEG-8 Caprylic/Capric Glycerides, PEG-8 Castor Oil, PEG-8 Glyceryl Laurate, PEG-8 Hydrogenated Castor Oil, PEG-8 Hydrogenated Fish Glycerides, PEG-9 Castor Oil, PEG-9 Cocoglycerides, Polyglyceryl-10 Caprate, Polyglyceryl-10 Decahydroxystearate, Polyglyceryl-10 Decaisostearate, Polyglyceryl-10 Decalinoleate, Polyglyceryl-10 Decamacadamiate, Polyglyceryl-10 Decaoleate, Polyglyceryl-10 Decastearate, Polyglyceryl-10 Didecanoate, Polyglyceryl-10 Diisostearate, Polyglyceryl-10 Dioleate, Polyglyceryl-10 Dipalmitate, Polyglyceryl-10 Distearate, Polyglyceryl-10 Dodecacaprate, Polyglyceryl-10 Dodecacaprylate, Polyglyceryl-10 Heptaoleate, Polyglyceryl-10 Heptastearate, Polyglyceryl-10 Hexaoleate, Polyglyceryl-10 Hydroxystearate/Stearate/Eicosadioate, Polyglyceryl-10 Isostearate, Polyglyceryl-10 Mono/Dioleate, Polyglyceryl-10 Nonaerucate, Polyglyceryl-10 Nonaisostearate, Polyglyceryl-10 Palm itate, Polyglyceryl-10 Pentahydroxystearate, Polyglyceryl-10 Pentaisostearate, Polyglyceryl-10 Pentalaurate, Polyglyceryl-10 Pentalinoleate, Polyglyceryl-10 Pentaoleate, Polyglyceryl-10 Pentaricinoleate, Polyglyceryl-10 Pentastearate, Polyglyceryl-10 Tetraoleate, Polyglyceryl-10 Tridecanoate, Polyglyceryl-10 Tristearate, Polyglyceryl-2 Caprate, Polyglyceryl-2 Caprylate, Polyglyceryl-2 Diisostearate, Polyglyceryl-2 Dioleate, Polyglyceryl-2 Isopalmitate, Polyglyceryl-2 Isostearate, Polyglyceryl-2 Laurate, Polyglyceryl-2 Myristate, Polyglyceryl-2 Oleate, Polyglyceryl-2 Palmitate, Polyglyceryl-2 Sesquicaprylate, Polyglyceryl-2 Sesquiisostearate, Polyglyceryl-2 Sesquioleate, Polyglyceryl-2 Sesquistearate, Polyglyceryl-2 Stearate, Polyglyceryl-2 Tetraisostearate, Polyglyceryl-2 Tetrastearate, Polyglyceryl-2 Triisostearate, Polyglyceryl-3 Caprate, Polyglyceryl-3 Dicaprate, Polyglyceryl-3 Dicocoate, Polyglyceryl-3 Di-Hydroxystearate, Polyglyceryl-3 Diisostearate, Polyglyceryl-3 Dioleate, Polyglyceryl-3 Distearate, Polyglyceryl-3 Isostearate, Polyglyceryl-3 Laurate, Polyglyceryl-3 Methylglucose Distearate, Polyglyceryl-3 Myristate, Polyglyceryl-3 Oleate, Polyglyceryl-3 Palmitate, Polyglyceryl-3 Pentaricinoleate, Polyglyceryl-3 Ricinoleate, Polyglyceryl-3 Stearate, Polyglyceryl-3 Triisostearate, Polyglyceryl-4 Caprate, Polyglyceryl-4 Dilaurate, Polyglyceryl-4 Isostearate, Polyglyceryl-4 Laurate, Polyglyceryl-4 Oleate, Polyglyceryl-4 Pentaoleate, Polyglyceryl-4 Pentastearate, Polyglyceryl-4 Tristearate, Polyglyceryl-5 Caprate, Polyglyceryl-5 Hexastearate, Polyglyceryl-5 Isostearate, Polyglyceryl-5 Laurate, Polyglyceryl-5 Myristate, Polyglyceryl-5 Oleate, Polyglyceryl-5 Pentamyristate, Polyglyceryl-5 Tribehenate, Polyglyceryl-5 Trimyristate, Polyglyceryl-5 Trioleate, Polyglyceryl-6 Caprylate, Polyglyceryl-6 Dicaprate, Polyglyceryl-6 Diisostearate, Polyglyceryl-6 Dioleate, Polyglyceryl-5 Dipalmitate, Polyglyceryl-6 Distearate, Polyglyceryl-6 Hexaoleate, Polyglyceryl-6 Hexastearate, Polyglyceryl-6 Isostearate, Polyglyceryl-6 Laurate, Polyglyceryl-6 Myristate, Polyglyceryl-6 Octastearate, Polyglyceryl-6 Oleate, Polyglyceryl-6 Palmitate, Polyglyceryl-6 Pentaoleate, Polyglyceryl-6 Pentaricinoleate, Polyglyceryl-6 Pentastearate, Polyglyceryl-6 Polyricinoleate, Polyglyceryl-6 Stearate, Polyglyceryl-6 Tetrabehenate, Polyglyceryl-6 Tetraoleate, Polyglyceryl-8 Decaerucate/Decaisostearate/Decaricinoleate, PPG-3 Hydrogenated Castor Oil, PPG-5 Lanolin Wax Glyceride, PPG-67 Glyceryl Ether, PPG-70 Glyceryl Ether, Rapeseed Glyceride, Rapeseed Glycerides, Rubus Occidentalis Seed Oil, Sunflower Seed Oil Glyceride, Sunflower Seed Oil Glycerides, Tall Oil Glycerides, Tallow Glycerides, Tribenzoyl Triricinolein, Triisostearoyl Polyglyceryl-3 Dimer Dilinoleate, Vegetable Glycerides Phosphate, Wheat Germ Glycerides.

Alkoxylated alcohol esters, for example, C12-15 Pareth-3 Benzoate, Cetyl C12-15 Pareth-8 Carboxylate, Di-PPG-2 Myreth-10 Adipate, Di-PPG-3 Ceteth-4 Adipate, Di-PPG-3 Myristyl Ether Adipate, Glycereth-5 Lactate, Glycereth-7 Benzoate, Glycereth-7 Diisononanoate, Glycereth-7 Lactate, Oleth-2 Benzoate, PEG-3 Butylene Glycol Laurate, PEG-5 Trilauryl Citrate, PEG-5 Trimyristyl Citrate, PEG-5 Tristearyl Citrate, PEG-6 Butylene Glycol Laurate, PEG-6 Propylene Glycol Caprylate/Caprate, PEG-8 Propylene Glycol Cocoate, PPG-15 Stearyl Ether Benzoate, PPG-20 Methyl Glucose Ether Distearate, PPG-3 Myristyl Ether Neoheptanoate, Methyl Gluceth-20 Benzoate.

Alkoxylated alcohol glyceryl esters and derivatives, for example, Glycereth-18 Benzoate, Glycereth-20 Benzoate, Glycereth-7 Triacetate, PEG-10 Castor Oil, PEG-10 Hydrogenated Castor Oil, PEG-10 Olive Glycerides, PEG-10 Sunflower Glycerides, PEG-11 Avocado Glycerides, PEG-11 Babassu Glycerides, PEG-11 Castor Oil, PEG-11 Cocoa Butter Glycerides, PEG-12 Glyceryl Dioleate, PEG-12 Glyceryl Distearate, PEG-12 Palm Kernel Glycerides, PEG-13 Mink Glycerides, PEG-13 Sunflower Glycerides, PEG-14 Avocado Glycerides, PEG-15 Castor Oil, PEG-15 Glyceryl Laurate, PEG-16 Castor Oil, PEG-16 Hydrogenated Castor Oil, PEG-16 Macadamia Glycerides, PEG-18 Glyceryl Oleate/Cocoate, PEG-18 Palm Glycerides, PEG-192 Apricot Kernel Glycerides, PEG-2 Castor Oil, PEG-2 Hydrogenated Castor Oil, PEG-2 Olive Glycerides, PEG-2 Sunflower Glycerides, PEG-20 Almond Glycerides, PEG-20 Corn Glycerides, PEG-20 Evening Primrose Glycerides, PEG-20 Glyceryl Stearate, PEG-20 Hydrogenated Palm Glycerides, PEG-25 Moring a Glycerides, PEG-3 Castor Oil, PEG-3 Glyceryl Cocoate, PEG-4 Castor Oil, PEG-4 Glyceryl Distearate, PEG-40 Olive Glycerides, PEG-42 Babassu Glycerides, PEG-42 Mushroom Glycerides, PEG-45 Palm Kernel Glycerides, PEG-45 Safflower Glycerides, PEG-5 Castor Oil, PEG-5 Hydrogenated Castor Oil, PEG-5 Hydrogenated Corn Glycerides, PEG-50 Glyceryl Triisostearate, PEG-6 Almond Glycerides, PEG-6 Glyceryl Caprate, PEG-60 Almond Glycerides, PEG-60 Corn Glycerides, PEG-60 Shea Butter Glycerides, PEG-65 Hydrogenated Castor Oil, PEG-7 Glyceryl Cocoate, PEG-7 Glyceryl Laurate, PEG-7 Glyceryl Soyate, PEG-7 Hydrogenated Castor Oil, PEG-7 Olive Glycerides, PEG-7 Sunflower Glycerides, PEG-70 Mango Glycerides, PEG-75 Cocoa Butter Glycerides, PEG-75 Meadowfoam Oil, PEG-75 Shea Butter Glycerides, PEG-75 Shorea Butter Glycerides, PEG-75 Soy Glycerides, PEG-8 Castor Oil, PEG-8 Glyceryl Laurate, PEG-8 Hydrogenated Castor Oil, PEG-8 Hydrogenated Fish Glycerides, PEG-9 Castor Oil, PEG-9 Cocoglycerides, PPG-3 Hydrogenated Castor Oil, PPG-5 Lanolin Wax Glyceride.

Alkoxylated alcohol ethers, for example, PPG-9 Diglyceryl Ether Glycereth-7 Trimethyl Ether, PPG-10 Cetyl Ether, PPG-10 Oleyl Ether, PPG-11 Stearyl Ether, PPG-14 Butyl Ether, PPG-15 Stearyl Ether, PPG-20 Cetyl Ether, PPG-20 Oleyl Ether, PPG-23 Oleyl Ether, PPG-28 Cetyl Ether, PPG-3 Benzyl Ether Myristate, PPG-3 Myristyl Ether, PPG-30 Cetyl Ether, PPG-30 Isocetyl Ether, PPG-30 Oleyl Ether, PPG-37 Oleyl Ether, PPG-3-Isodeceth-1, PPG-4 Lauryl Ether, PPG-4 Myristyl Ether, PPG-5 Pentaerythrityl Ether, PPG-50 Cetyl Ether, PPG-50 Oleyl Ether, PPG-65-PEG-5 Pentaerythrityl Ether, PPG-9-Steareth-3.

Fats and oils, for example, fats and oils, as a group, are the glyceryl esters of fatty acids (triglycerides) normally found in animal and plant tissues, including those which have been hydrogenated to reduce or eliminate unsaturation. Also included are synthetically-prepared esters of glycerin and fatty acids. They possess the class:

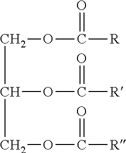

in which —COR, —COR′, —COR″ may be the same or different fatty acid radicals.

Naturally occurring fats and oils in this group are esters and can be hydrolyzed to yield glycerin and mixtures of fatty acids. These fatty acids may serve as raw materials for a variety of other chemicals useful in cosmetics. Alternately, isolated and purified fatty acids can be esterified with glycerin to yield mono-, di-, and triglycerides. Fats and oils are used directly in cosmetics as emollient vehicles and lubricants. Volatile oils obtained by the distillation of plant material are classified as essential oils.

Fats and oils with occlusive properties: Occlusive are cosmetic ingredients that slow down the evaporation of water from the skin surface. This function is different from antiperspirant agents that interfere with the delivery of liquid water to the skin surface. By blocking the evaporative loss of water, occlusive materials increase the water content of skin. Occlusive agents are generally lipids that tend to remain on the skin surface. Examples of fats and oils with occlusive properties are:

Argania Spinosa Kernel Oil, Argemone Mexicana Oil, Astrocaryum Vulgare Fruit Oil, Astrocaryum Vulgare Kernel Oil, Avena Sativa (Oat) Kernel Oil, Balanites Roxburghii Seed Oil, Bassia Butyracea Seed Butter, Bassia Latifolia Seed Butter, Brassica Campestris (Rapeseed) Seed Oil, Buffalo Fat, Butter, Butyrospermum Parkii (Shea Butter), Butyrospermum Parkii (Shea Butter) Oil, C10-18 Triglycerides, C12-18 Acid Triglyceride, C8-12 Acid Triglyceride, Calendula Officinalis Seed Oil, Calophyllum Tacamahaca Seed Oil, Camellia Japonica Seed Oil, Camellia Kissi Seed Oil, Camellia Sinensis Seed Oil, Canola Oil, Caprylic/Capric Triglyceride, Caprylic/Capric/Lauric Triglyceride, Caprylic/Capric/Linoleic Triglyceride, Caprylic/Capric/Myristic/Stearic Triglyceride, Caprylic/Capric/Stearic Triglyceride, Carthamus Tinctorius (Safflower) Seed Oil, Carya Illinoensis (Pecan) Seed Oil, Caryocar Coriaceum Seed Oil, Castor Oil Benzoate, Cibotium Barometz Oil, Citrullus Vulgaris (Watermelon) Seed Oil, Cocos Nucifera (Coconut) Oil, Cod Liver Oil, Cod Liver/Mink/Tallow Triglyceride, Coix Lacryma-Jobi (Job's Tears) Seed Oil, Corylus Americana (Hazel) Seed Oil, Crambe Abyssinica Seed Oil, Cucurbita Pepo (Pumpkin) Seed Oil, Deer Fat, Elaeis Guineensis (Palm) Kernel Oil, Elaeis Guineensis (Palm) Oil, Emu Oil, Euterpe Oleracea Fruit Oil, Fish Liver Oil, Garcinia Indica Seed Butter, Gevuina Avellana Oil, Glycine Soja (Soybean) Lipids, Glycine Soja (Soybean) Oil, Goat Butter, Goat Fat, Gossypium Herbaceum (Cotton) Seed Oil, Helianthus Annuus (Sunflower) Seed Oil, Hippophae Rhamnoides Oil, Horse Fat, Hydrogenated Apricot Kernel Oil, Hydrogenated C12-18 Triglycerides, Hydrogenated Canola Oil, Hydrogenated Castor Oil, Hydrogenated Castor Oil Laurate, Hydrogenated Castor Oil Triisostearate, Hydrogenated Coconut Oil, Hydrogenated Cottonseed Oil, Hydrogenated Fish Oil, Hydrogenated Lard, Hydrogenated Menhaden Oil, Hydrogenated Milk Lipids, Hydrogenated Mink Oil, Hydrogenated Olive Oil, Hydrogenated Orange Roughy Oil, Hydrogenated Ostrich Oil, Hydrogenated Palm Kernel Oil, Hydrogenated Palm Oil, Hydrogenated Peanut Oil, Hydrogenated Pistachio Seed Oil, Hydrogenated Rapeseed Oil, Hydrogenated Rice Bran Oil, Hydrogenated Safflower Seed Oil, Hydrogenated Shark Liver Oil, Hydrogenated Shea Butter, Hydrogenated Soybean Oil, Hydrogenated Sunflower Seed Oil, Hydrogenated Sweet Almond Oil, Hydrogenated Tallow, Hydrogenated Vegetable Oil, Hydrogenated Wheat Germ Oil, Irvingia Gabonensis Kernel Butter, Isatis Tinctoria Seed Oil, Juglans Regia (Walnut) Seed Oil, Kyounin Yu, Lard, Lauric/Palmitic/Oleic Triglyceride, Limnanthes Alba (Meadowfoam) Seed Oil, Linum Usitatissimum (Linseed) Seed Oil, Luffa Cylindrica Seed Oil, Lupinus Albus Seed Oil, Macadamia Integrifolia Seed Oil, Macadamia Ternifolia Seed Oil, Mangifera Indica (Mango) Seed Butter, Mangifera Indica (Mango) Seed Oil, Marmot Oil, Melia Azadirachta Seed Oil, Melissa Officinalis Seed Oil, Menhaden Oil, Mink Oil, Moring a Pterygosperma Seed Oil, Mortierella Oil, Natane Yu, Neatsfoot Oil, Nigella Sativa Seed Oil, Oenocarpus Bacaba Fruit Oil, Oenocarpus Bataua Fruit Oil, Olea Europaea (Olive) Fruit Oil, Olea Europaea (Olive) Husk Oil, Oleic/Linoleic Triglyceride, Oleic/Palmitic/Lauric/Myristic/Linoleic Triglyceride, Omental Lipids, Orange Roughy Oil, Orbignya Cohune Seed Oil, Orbignya Oleifera Seed Oil, Oryza Sativa (Rice) Bran Oil, Oryza Sativa (Rice) Germ Oil, Ostrich Oil, Papaver Orientale (Poppy) Seed Oil, Passiflora Edulis Seed Oil, Persea Gratissima (Avocado) Butter, Persea Gratissima (Avocado) Oil, Pinus Koraiensis Seed Oil, Pistacia Vera Seed Oil, Prunus Amygdalus Dulcis (Sweet Almond) Oil, Prunus Armeniaca (Apricot) Kernel Oil, Prunus Avium (Sweet Chemy) Seed Oil, Prunus Domestica Seed Oil, Prunus Insititia Seed Oil, Prunus Persica (Peach) Kernel Oil, Rabbit Fat, Ricinoleic/Caproic/Caprylic/Capric Triglyceride, Ricinus Communis (Castor) Seed Oil, Rosa Eglentaria Seed Oil, Salmon Oil, Salvia Hispanica Seed Oil, Santalum Album (Sandalwood) Seed Oil, Schleichera Trijuga Seed Oil, Sesamum Indicum (Sesame) Seed Oil, Shark Liver Oil, Shorea Robusta Seed Butter, Solanum Lycopersicum (Tomato) Fruit Oil, Solanum Lycopersicum (Tomato) Seed Oil, Tall Oil, Tallow, Telphairia Pedata Oil, Theobroma Cacao (Cocoa) Seed Butter, Theobroma Grandiflorum Seed Butter, Triarachidin, Tribehenin, Tricaprin, Tricaprylin, Trichodesma Zeylanicum Oil, Trierucin, Triethylhexanoin, Triheptanoin, Triheptylundecanoin, rihydroxymethoxystearin, Trihydrooxystearin, Triisononanoin, Triisopalmitin, Triisostearin, Trilaurin, Trilinolein, Trilinolenin, Trimyristin, Triolein, Tripalmitin, Tripalmitolein, Triricinolein, Trisebacin, Tristearin, Triticum Vulgare (Wheat) Bran Lipids, Triticum Vulgare (Wheat) Germ Oil, Triundecanoin, Turtle Oil, Vaccinium Macrocarpon (Cranberry) Seed Oil, Vateria Indica Seed Butter, Virola Sebifera Nut Oil, Zea Mays (Corn) Germ Oil, Zea Mays (Corn) Oil.

Fats and oils with emollient properties: Emollient are cosmetic ingredients that help to maintain the soft, smooth, and pliable appearance of skin. Emollients function, via their ability to remain on the skin surface or in the stratum corneum, and act as lubricants to reduce flaking, and improve the skin's appearance. Examples of fats and oils with emollient properties are:

Acetylated Hydrogenated Castor Oil, Actinidia Chinensis (Kiwi) Seed Oil, Adansonia Digitata Seed Oil, Argania Spinosa Kernel Oil, Argemone Mexicana Oil, Astrocaryum Murumuru Butter, Bertholletia Excelsa Seed Oil, Bixa Orellana Seed Oil, Borago Officinalis Seed Oil, Brain Lipids, Brassica Oleracea Italica (Broccoli) Seed Oil, C10-40 Isoalkyl Acid Triglyceride, Calodendrum Capense Nut Oil, Calophyllum Tacamahaca Seed Oil, Camellia Kissi Seed Oil, Carica Papaya Seed Oil, Caryocar Coriaceum Seed Oil, Castor Oil Benzoate, Chenopodium Quinoa Seed Oil, Citrullus Lanatus (Watermelon) Seed Oil, Cod Liver/Mink/Tallow Triglyceride, Coix Lacryma-Jobi (Job's Tears) Seed Oil, Cucumis Sativus (Cucumber) Oil, Daucus Carota Sativa (Carrot) Seed Oil, Glyceryl Triacetyl Hydroxystearate, Glyceryl Triacetyl Ricinoleate, Haematococcus Pluvialis Oil, Hydrogenated Egg Oil, Hydrogenated Evening Primrose Oil, Hydrogenated Grapeseed Oil, Hydrogenated Hazelnut Oil, Hydrogenated Hemp Oil, Hydrogenated Hemp Seed Oil, Hydrogenated Macadamia Seed Oil, Hydrogenated Sesame Seed Oil, Isatis Tinctoria Seed Oil, Lithospermum Officinale Seed Oil, Lupinus Albus Seed Oil, Lycium Barbarum Seed Oil, Mamushi Oil, Marrow Lipids, Milk Lipids, Morinda Citrifolia Seed Oil, Mucor Circinelloides Oil, Mustelic/Palmitic Triglyceride, Odontella Aurita Oil, Orbignya Speciosa Kernel Oil, Oxycoccus Palustris Seed Oil, Papaver Somniferum Seed Oil, Parinari Curatellifolia Seed Oil, Passiflora Edulis Seed Oil, Pinus Parviflora Seed Oil, Pinus Pentaphylla Seed Oil, Plukenetia Volubilis Seed Oil, Prunus Insititia Seed Oil, Punica Granatum Seed Oil, Pycnanthus Angolensis Nut Oil, Pyrus Malus (Apple) Seed Oil, Ribes Nigrum (Black Currant) Seed Oil, Rosa Moschata Seed Oil, Rosa Rubiginosa Seed Oil, Rubus Glaucus Seed Oil, Rubus Idaeus (Raspberry) Seed Oil, Schinziophyton Rautanenii Kernel Oil, Sclerocarya Birrea Seed Oil, Silkworm Chrysalis Oil, Soybean Glycerides, Spinal Cord Lipids, Sterculia Africana Seed Oil, Torreya Nucifera Seed Oil, Vaccinium Vitis-Idaea Seed Oil, Vanilla Planifolia Fruit Oil, Virola Sebifera Nut Oil.

Further preferred lipophilic emollients are fatty alcohols. Fatty alcohols are higher molecular weight nonvolatile alcohols. They are produced from natural fats and oils by reduction of the fatty acid —COOH grouping to the hydroxyl function. Alternately, several completely synthetic routes yield fatty alcohols that may be structurally identical or similar to the nature-derived alcohols. Fatty alcohols generally are primary alcohols conforming to the class RCH₂OH; those fatty alcohols prepared from naturally occurring fatty acids normally contain an even number of carbon atoms. Fatty alcohols are used as emollients in numerous types of cosmetics. They are valuable as co-emulsifiers and are employed to increase the viscosity of emulsions, shampoos, and other products. In addition, these alcohols are used for the synthesis of some widely used cosmetic ingredients (e.g., alkoxylated alcohols, alkyl sulfates, alkyl ether sulfates, and phosphorus compounds). Further examples are: Arachidyl Alcohol, Behenyl Alcohol, C9-11 Alcohols, C12-13 Alcohols, C12-15 Alcohols, C12-16 Alcohols, C14-15 Alcohols, C14-22 Alcohols, C20-22 Alcohols, Caprylic Alcohol, Cetearyl Alcohol, Cetyl Alcohol, Coconut Alcohol, C6-12 Perfluoroalkylethanol, Decyl Alcohol, Hydrogenated Jojoba Alcohol, Hydrogenated Rapeseed Alcohol, Hydrogenated Tallow Alcohol, Jojoba Alcohol, Lauryl Alcohol, Myristyl Alcohol, Oleyl Alcohol, Olive Alcohol, Palm Alcohol, Palm Kernel Alcohol, Stearyl Alcohol, Tallow Alcohol, Tridecyl Alcohol.

Further preferred lipophilic emollients are ethers. Ethers are the organic compounds possessing a C—O—C system of bonds. If this C—O—C system is part of a ring, the corresponding compound is classified with the heterocyclics, e.g., tocopherol. Sorbitan derivatives are also heterocyclic ethers. Most ethers used in cosmetics are prepared synthetically. Alkoxylated derivatives are ethers, but are herein classed as alkoxylated alcohols, alkoxylated amides, alkoxylated amines, alkoxylated carboxylic acids, and polymeric ethers. The esterified polyglyceryl ethers resulting from the dehydration of glycerin are included in the group of glyceryl esters.

Ethers with emollient properties are cosmetic ingredients that help to maintain the soft, smooth, and pliable appearance of skin. Emollients function, via their ability to remain on the skin surface or in the stratum corneum, and act as lubricants to reduce flaking, and improve the skin's appearance. Examples of such compounds are: Butoxyethyl Stearate, C12-14 Hydroxyalkyl Hydroxydimerdilinoleyl Ether, C12-14 Hydroxyalkyl Maltitol Ether, C16-18 Hydrooxyalkyl Hydroxydimerdilinoleyl Ether, Cetyl Glyceryl Ether, Cetyloxypropyl Glyceryl Methoxypropyl Myristamide, Chimyl Alcohol, Chimyl Isostearate, Chimyl Stearate, Dicaprylyl Ether, Diretinyl Ether, Ethoxydiglycol Behenate, Ethoxydiglycol Isostearate, Ethoxydiglycol Oleate, Ethylhexyloxyglyceryl Palmitate, Eugenyl Glucoside, Hydrolyzed Proanthocyanidin, Hydroxyethyl Palmityl Oxyhydroxypropyl Palmitamide, lsopolyglycerin-3 Dimethicone, Isopropyl Hydroxycetyl Ether, Isostearyl Ferulate, Isostearyl Glyceryl Ether, Isostearyl Glyceryl Pentaerythrityl Ether, Lauryl Polyglyceryl-6 Cetearyl Glycol Ether, Oleyl Glyceryl Ether, Polyglyceryl-2 Lanolin Alcohol Ether, Polyglyceryl-2 Oleyl Ether, Polyglyceryl-3 Decyltetradecyl Ether, Polyglyceryl-3 Hydroxylauryl Ether, Polyglyceryl-4 Lauryl Ether, Polyglyceryl-4 Oleyl Ether, PPG-9 Diglyceryl Ether, Propylene Glycol Myristyl Ether.

Ethers with solvent properties are, for exmaple, Butoxyethyl Acetate, Diethoxydiglycol, Dimethoxydiglycol, Dimethyl Ether, Dimethyl Isosorbide, Dioxolane, Dipropylene Glycol, Dipropylene Glycol Dimethyl Ether, Ethoxydiglycol Acetate, Ethoxyethanol Acetate, Ethyl Ether, Ethyl Perfluorobutyl Ether, Ethyl Perfluoroisobutyl Ether, Methoxydiglycol, Methoxyethanol Acetate, Methoxyisopropyl Acetate, Methyl Hexyl Ether, Methyl Perfluorobutyl Ether, Methyl Perfluoroisobutyl Ether, Methylal, Propylene Glycol t-Butyl Ether.

Further preferred lipophilic emollients are hydrocarbons. Hydrocarbons are the group of compounds containing only carbon and hydrogen. Hydrocarbons are generally derived from petrochemicals, but some of them are found in the plant and animal kingdom (e.g., azulene or squalene). Their class can vary widely, and include aliphatic, alicyclic, and aromatic compounds. This category also includes some materials of complex composition which can be a source of a variety of hydrocarbons.

As a rule these compounds are chemically inert. Typical are petrolatum, paraffin, and mineral oil, all of which are widely used as emollients and vehicles in cosmetics because of their nonvolatility and unctuous character. Lower molecular weight volatile hydrocarbons are used as solvents (e.g., mineral spirits). The hydrocarbons which are normally gases at room temperature (e.g., butane or propane) are used as aerosol propellants. Finally, it should be noted that many synthetic polymers are hydrocarbons (e.g., polyethylene).

Hydrocarbons with emollient properties are cosmetic ingredients that help to maintain the soft, smooth, and pliable appearance of skin. Emollients function, via their ability to remain on the skin surface or in the stratum corneum, and act as lubricants to reduce flaking, and improve the skin's appearance. Examples of such compounds are: C12-20 Isoparaffin, C20-40 Isoparaffin, C9-16 Isoparaffin, Eicosane, Hydrogenated Polydecene, Hydrogenated Polydodecene, Hydrogenated Polyisobutene, lsoeicosane, Isohexadecane, Mineral Oil, Octadecane, Squalene.

Hydrocarbons with occlusive properties are cosmetic ingredients that slow down the evaporation of water from the skin surface. This function is different from antiperspirant agents that interfere with the delivery of liquid water to the skin surface. By blocking the evaporative loss of water, occlusive materials increase the water content of skin. Occlusive agents are generally lipids that tend to remain on the skin surface. Examples of such compounds are: C18-26 Olefin, C18-70 Isoparaffin, C20-24 Olefin, C24-28 Olefin, C24-30 Olefin, C26-54 Olefin, C28-36 Olefin, C30-45 Olefin, C4-12 Polyolefin, Didecene, Diethylhexylcyclohexane, Mineral Oil, Paraffin, Pentahydrosqualene, Petrolatum, Pristane, Squalane.

Hydrocarbons with solvent properties are, for example, C10-11 Isoparaffin, C10-12 Isoparaffin, C11-12 Isoparaffin, C11-13 Isoparaffin, C11-14 Isoparaffin, C12-14 Isoparaffin, C13-14 Isoparaffin, C13-16 Isoparaffin, Decane, Decene, Dodecene, Heptane, Hexadecene, Hexane, Isododecane, Limonene, Octadecene, Octene, Petroleum Distillates, Tetradecene.

Further preferred lipophilic emollients are silicones. As generally accepted by “silicones” is intended any organosilicon polymers or oligomers having a linear or cyclic, branched or crosslinked structure, of variable molecular weight, and essentially based of recurring structural units in which the silicone atoms are linked to each other by oxygen atoms (siloxane bond SiOSi), optionally substituted hydrocarbon radicals being directly linked via a carbon atom to the silicone atoms. Examples are:
Cyclic siloxane polymers; cyclomethicones such as cyclopentasiloxane, cyclohexasiloxane,

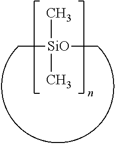

The INCI names for labeling specific cyclic dimethyl polysiloxane compounds are: Cyclotrisiloxane (q.v.) when n is equal to 3, Cyclotetrasiloxane (q.v.) when n is equal to 4, Cyclopentasiloxane (q.v.) when n is equal to 5, Cyclohexasiloxane (q.v.) when n is equal to 6, and Cycloheptasiloxane when n is equal to 7 (q.v.).
Linear siloxane polymer end-blocked with trimethylsiloxy units (M unit); dimethicones, non polar liquid with broad range of viscosity.
Silanol compounds or dimethiconols; dimethyl siloxane terminated with hydroxyl groups.
Alkyl-modified siloxanes (AMS). AMS improve spreadability and wash-off resistance.
Alkyl dimethicones and alkyl methicones, for example, bis-phenylpropyl dimethicone (SF 1555 fluid; GE Silicone), silicone waxes such as DC 2503 cosmetic wax (Dow Corning); Stearyl Dimethicone, DC 2502 fluid (Dow Corning); Cetyl Dimethicone, DC AMS-C30 wax (Dow Corning); C30-C45 Alkyl Methicone, DC 580 wax (Dow Corning), Stearoxytrimethylsilane and Stearyl Alcohol

An active agent, active agent composition or active agent extract suitable for skin cosmetics is an ingredient or a mixture of ingredients which is approved for dermal or topic administration. Examples to be listed are:
active agents having a deodorising and antiperspirant action: these include antiperspirants based on aluminium or zinc salts, deodorants containing bactericidal or bacteriostatic deodorising substances, for example triclosan, hexachlorophene, alcohols and cationic substances, for example quaternary ammonium salts and smell absorbers, for example ®Grillocin (combination of zinc rizinoleate and different additives) or triethylcitrates, optionally in combination with an antioxidant, such as butylhydroxytoluene), or ion exchange resins;
insect repellents: repellents are compositions which should prevent insects from touching the skin and getting active there. They repel the insects and slowly evaporate. The most frequently used repellent is dethyltoluamide (DEET). Other customary repellents are to be found in “W. Raab and U. Kindl, “Pflegekosmetik”, Gustav-Fischer-Verlag Stuttgart/New York, 1991, p. 161;
active agents protecting against chemical and mechanical attack: these include all those substances which form a barrier between the skin and the outer noxa, such as paraffin oils, silicone oils, vegetable oils, PCL products and lanoline as protection against aqueous solutions, film formers, such as sodium alginate, triethanolamine alginate, polyacrylates, polyvinyl alcohol or cellulose ethers against the action of organic solvents, or substances based on mineral oils, vegetable oils or silicone oils as “lubricants” against high mechanical stresses of the skin;
moisturising substances: the following substances are used as moisturisers: sodium lactate, urea, alcohols, sorbitol, glycerol, propylene glycol, collagen, elastin or hyaluronic acid;
active agents having ceratoplastic activity: benzoylperoxide, retinic acid, colloidal sulfur and resorcinol;
antimicrobial agents, for example triclosan or quaternary ammonium compounds;
dermally applicable oily or oil-soluble vitamins or vitamin derivatives: e.g. vitamin A (getinol in the form of the free acid or its derivatives), panthenol, pantothenic acid, folic acid, and combinations thereof, vitamin E (tocopherol), F; essential fatty acids; or niacinamide (nicotinic acid amide);
vitamin-based placenta extracts: active agent compositions mainly with vitamin A, C, E, B₂₁ B₁₂, folic acid and biotin, amino acids and enzymes and also compounds of the trace elements magnesium, silicium, phosphorus, calcium, manganese, iron or copper.
skin repair complexes: obtainable from inactivated and desintegrated cultures of bacteria of the bifidus group;
plants and plant extracts: for example arnica, aloe, beard lichen, ivy, nettle, ginseng, henna, camomile, calendula, rosemary, sage, equisetum or thyme;
animal extracts: for example royal jelly, propolis, proteins or thymus extracts;
dermally applicable cosmetic oils: neutral oils of the miglyol 812 type, apricot kernel oil, avocado oil, babassu oil, cottonseed oil, borage oil, thistle oil, groundnut oil, gammaoryzanol, rosehip seed oil, hemp oil, hazelnut oil, currant seed oil, jojoba oil, cherrystone oil, salmon oil, linseed oil, corn oil, macadamia nut oil, almond oil, evening primrose oil, mink oil, olive oil, pecan nut oil, peach kernel oil, pistachio nut oil, rapeseed oil, rice germ oil, castor oil, safflower oil, sesame oil, soybean oil, sunflower oil, tea tree oil, grapeseed oil or wheat germ oil.

Preferably, component (c) comprises esters represented by the class RCO—OR′, wherein RCO— represents the carboxylic acid radical and —OR′ is the alcohol residue; glyceryl esters and derivatives, alkoxylated alcohol esters, alkoxylated alcohol glyceryl esters and derive-tives, alkoxylated alcohol ethers, fats and oils, ethers, hydrocarbons or silicones, or a mixture of these substances.

Component (c) is present in the nanodispersions used according to this invention in a concentration of preferably 0.1 to 80% by weight, based on the total weight of components (a), (b), (c) and (d).

The nanodispersion used according to this invention optionally comprises as facultative component (f) a non aqueous polar organic solvent to fasten and improve the hydration rate of component (a). A variety of non aqueous polar organic solvent are suitable for use in the discontinuous phase of the nanoemulsion, described in this invention. Examples are as follows:
polyols that are defined as compounds which contain three or more hydroxyl groups per molecule. Examples of suitable polyols include fructose, glucamine, glucose, glucose glutamate, glucuronic acid, glycerine, 1,2,6 hexanetriol, hydroxystearyl methylglucamine, inositol, lactose, malitol, mannitol, methyl gluceth-10, methyl gluceth-20, methylglucose dioleate, methyl glucose sesquicocoate, methyl glucose sesquiisostearate, methyl glucose sesquilaurate, methyl glucose sesquistearate, phytantriol, riboflavin, sorbeth-6, sorbeth-20, sorbeth-30, sorbeth-40, sorbitol, sucrose, thioglycerin, xylitol, and mixtures thereof. A preferred polyol is glycerin.
Homopolymeric or block copolymeric liquid ethers which are preferably formed by polymerization of monomeric alkylene oxides, generally ethylene or propylene oxides. Preferred monomeric ethers are those exhibiting the structure below where n=1. Preferred polymeric ethers exhibit the general structure below wherein n=2 to 20:

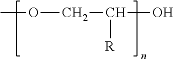

where R is hydrogen or C₁₋₁₀ straight or branched chain alkyl, and n is 1 to 20. Examples of such polymeric ethers include PEG, PPG, and derivatives thereof.

Other examples of suitable polymeric ethers include polyoxypropylene polyoxyethylene block copolymers having the general formula:

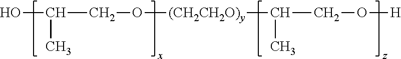

wherein x is 1 to 200, y is 1 to 200 and z is 1 to 200. Such compounds are available under the CTFA name Meroxapol® 105, 108, 171, 174, 178, 251, 252, 254, 255, 258, 311, 312 and 314.
mono- and dihydric alcohols of the general formula R(OH)ₙ where n is 1 or 2 and R is a substituted or unsubstituted saturated C2-C10, preferably C1-C8 alkyl, or a substituted or unsubstituted alicyclic, bicyclic, or aromatic ring, with the substituents selected from halogen, alkoxy, hydroxyl, and so on. Most suitable monohydric alcohols include ethanol, isopropanol, hexyldecanol, benzyl alcohol, propyl alcohol, and isopropyl alcohol. Most suitable dihydric alcohols such as hexylene glycol, diethylene glycol, ethylene glycol, ethylene glycol, propylene glycol, 1-2 butylene glycol, triethylene glycol, dipropylene glycol, and mixtures thereof.
sorbitan derivatives, which are defined as ethers or esters of sorbitan, are also suitable polar solvents such as the polysorbates, which are defined as stearate esters of sorbitol and sorbitan anhydrides; examples are polysorbate 20, 21, 40, 60, 61, 65, 80, 81 and 85.

Cosmetic end formulations include a very wide range of cosmetic products. Suitable products are, for example, especially the following:
skin-care products, for example skin washing and cleansing products in the form of bars of soap or liquid soaps, syndets or washing pastes;
bath products, for example liquid (foam baths, milks, shower products) or solid bath products, such as bath pearls and bath salts;
skin-care products, such as skin emulsions, multiple emulsions or skin oils;
decorative body-care products, for example face make-ups in the form of day or powder creams, face powders (lose and compressed), rouge or cream make-ups, eye-care products, for example eye shadow products, mascara, eyeliners, eye creams or eye-fix creams; lip-care products, for example lipstick, lip gloss, lip liner, nail-care products, such as nail varnish, nail varnish remover, nail hardeners or cuticle removers;
feminine hygiene products, such as feminine hygiene washing lotions or sprays;
foot-care products, for example foot baths, foot powders, food creams or foot balms, special deodorants and antiperspirants or products for scrubbing off callouses;
sunscreens, such as sun milks, lotions, creams, oils, sunblockers or tropicals, pre-sun products or after-sun products;
suntanning products, for example self-tanning creams;
depigmenting products, for example products for bleaching or lightening skin;
insect repellents, for example insect oils, lotions, sprays or sticks;
deodorants, for example deodorant sprays, non-aerosol sprays, deodorant gels, sticks or roll-ons;
antiperspirants, for example antiperspirant sticks, creams or roll-ons;
products for cleansing and treating impure skin, for example syndets (solid or liquid), peeling or scrubbing products or peeling masks;
chemical depilatory products, for example depilatory powders, liquid depilatory products, creamy or pasty depilatory products, depilatory gels or aerosol foams;
shaving products, for example shaving soap, foaming shaving creams, non-foaming shaving creams, shaving foams and gels, preshaving products for dry shaving, aftershaves or aftershave lotions;
scents, for example perfumes (Eau de Cologne, Eau de Toilette, Eau de Parfum, Parfum de Toilette, perfume), perfume oils or perfume creams;
products for oral and dental hygiene as well as for dentures, for example toothpastes, tooth gels, tooth powders, mouth-wash concentrates, anti-plaque mouth-washes, denture cleaning products or denture adhesion products;
cosmetic formulations for hair treatment, for example hair washes in the form of shampoos, hair conditioners, hair-care products, for example pretreatment products, hair tonics, hair styling creams and gels, pomades, hair rinses, deep conditioning treatments, intensive hair care treatments, hair setting products, for example waving agents for perms (hot wave, mild wave, cold wave), hair straightening products, liquid hair fixatives, hair foams, hair sprays, bleaching agents, for example hydrogen peroxide solutions, bleaching shampoos, bleaching creams, bleaching powders, bleaching pastes or oils, temporary, semitemporary or permanent hair dyes, products containing self-oxidising dyes, or natural hair dyes, such as henna or camomile.

Preferably, at least one component (a), (b) or (c) is an ingredient used in cosmetics for treating or protecting the skin, mucosae or hair.

The end formulations listed above can be in a very wide range of forms of presentation, for example
in the form of liquid formulations as an O/W emulsion,
in the form of a stick,
in the form of a gel,
in the form of an cream, lotion or milk,
in the form of a spray (spray with propellant or non-aerosol spray) or an aerosol,
in the form of a foam,
in the form of a paste, or
in the form of a powder, lacquer, pellet or make-up.

The cosmetic end formulation containing one or several of the above ingredients which may be in the form of the above-mentioned forms of presentation, contains the nanodispersion used according to this invention preferably in a concentration of 0.01 to 99, preferably of 0.01 to 20, more preferably of 0.05 to 10% by weight, based on the total weight of the cosmetic end-product

The following Examples illustrate the invention further. Parts or percentages relate to weight.

### EXAMPLE 1

| | Examples | | | | | | | | | |
|---|---|---|---|---|---|---|---|---|---|---|
| Component [% b.w.] | a | b | c | d | e | f | g | h | i | j |
| Co-emulsifier such as | 0.50 | 1.00 | 2.00 | 3.00 | 4.00 | 8.00 | 10.00 | 15.00 | 20.00 | 25.00 |
| Cetyl Dimethicone | | | | | | | | | | |
| Copolyol (INCI) | | | | | | | | | | |
| Co-emulsifier such as | 1.00 | 2.00 | 5.00 | 3.00 | 4.00 | 5.00 | 8.00 | 10.00 | 15.00 | 3.00 |
| Diisostearoyl polyglyceryl-3 | | | | | | | | | | |
| Dilinoleate (INCI) | | | | | | | | | | |
| Hydrogenated Castor oil | 1.00 | 2.00 | 1.00 | 2.00 | 2.00 | 1.00 | 2.00 | 1.00 | 2.00 | 1.00 |
| and/or microcrystalline | | | | | | | | | | |
| Wax (INCI) | | | | | | | | | | |
| Membrane-forming agent | 3.00 | 5.00 | 1.50 | 2.00 | 3.50 | 3.00 | 5.00 | 1.50 | 2.00 | 3.50 |
| such as phospholipid of | | | | | | | | | | |
| formula (1) as described | | | | | | | | | | |
| in claim 14 | | | | | | | | | | |
| Lipophilic component | 22.00 | 19.00 | 19.00 | 16.50 | 17.50 | 31.00 | 26.00 | 29.00 | 25.00 | 27.50 |
| such as Caprylic/Capric | | | | | | | | | | |
| Triglycerides (INCI) | | | | | | | | | | |
| Water-soluble active | 0.50 | 1.00 | 5.00 | 2.00 | 10.00 | 10.00 | 5.00 | 2.00 | 1.00 | 0.50 |
| ingredient such as | | | | | | | | | | |
| Ascorbic acid (INCI) | | | | | | | | | | |
| Antioxidant as defined in | 2.50 | 2.00 | 1.50 | 3.00 | 1.00 | 2.00 | 3.00 | 2.50 | 1.80 | 0.80 |
| claims 7, 8, 9 and 10 of | | | | | | | | | | |
| hereby invention such as | | | | | | | | | | |
| compound of formula A or B | | | | | | | | | | |
| Butylene Glycol | 2.50 | 1.50 | 2.00 | 3.00 | 3.50 | — | — | — | — | — |
| Sorbeth-20 | — | — | — | — | — | 2.50 | 1.50 | 2.00 | 3.00 | 3.50 |
| Water deionized | Qsp | Qsp | Qsp | Qsp | Qsp | Qsp | Qsp | Qsp | Qsp | Qsp |
| | 100 | 100 | 100 | 100 | 100 | 100 | 100 | 100 | 100 | 100 |

### EXAMPLE 2

| | Examples | | | | | | | | | |
|---|---|---|---|---|---|---|---|---|---|---|
| Component [% b.w.] | a | b | c | d | e | f | g | h | i | j |
| Co-emulsifier such as | 0.50 | 1.00 | 2.00 | 3.00 | 4.00 | 8.00 | 10.00 | 15.00 | 20.00 | 25.00 |
| PEG-9 polydimethylsil- | | | | | | | | | | |
| Oxyethyl dimethicone (INCI) | | | | | | | | | | |
| Co-emulsifier such as | 1.00 | 2.00 | 5.00 | 3.00 | 4.00 | 5.00 | 8.00 | 10.00 | 15.00 | 3.00 |
| Diisostearoyl polyglyceryl-3 | | | | | | | | | | |
| Dilinoleate (INCI) | | | | | | | | | | |
| Hydrogenated Castor oil | 1.00 | 2.00 | 1.00 | 2.00 | 2.00 | 1.00 | 2.00 | 1.00 | 2.00 | 1.00 |
| and/or microcrystalline | | | | | | | | | | |
| Wax (INCI) | | | | | | | | | | |
| Membrane-forming agent | 3.00 | 5.00 | 1.50 | 2.00 | 3.50 | 3.00 | 5.00 | 1.50 | 2.00 | 3.50 |
| such as phospholipid of | | | | | | | | | | |
| formula (1) as described | | | | | | | | | | |
| in claim 14 | | | | | | | | | | |
| Lipophilic component | 22.00 | 19.00 | 19.00 | 16.50 | 17.50 | 31.00 | 26.00 | 29.00 | 25.00 | 27.50 |
| such as Caprylic/Capric | | | | | | | | | | |
| Triglycerides (INCI) | | | | | | | | | | |
| Water-soluble active | 0.50 | 1.00 | 5.00 | 2.00 | 10.00 | 10.00 | 5.00 | 2.00 | 1.00 | 0.50 |
| ingredient such as | | | | | | | | | | |
| Ascorbic acid (INCI) | | | | | | | | | | |
| Antioxidant as defined in | 2.50 | 2.00 | 1.50 | 3.00 | 1.00 | 2.00 | 3.00 | 2.50 | 1.80 | 0.80 |
| claims 7, 8, 9 and 10 of | | | | | | | | | | |
| hereby invention such as | | | | | | | | | | |
| compound of formula A or B | | | | | | | | | | |
| Butylene Glycol | 2.50 | 1.50 | 2.00 | 3.00 | 3.50 | — | — | — | — | — |
| Sorbeth-20 | — | — | — | — | — | 2.50 | 1.50 | 2.00 | 3.00 | 3.50 |
| Water deionized | Qsp | Qsp | Qsp | Qsp | Qsp | Qsp | Qsp | Qsp | Qsp | Qsp |
| | 100 | 100 | 100 | 100 | 100 | 100 | 100 | 100 | 100 | 100 |

### EXAMPLE 3

| | Examples | | | | | | | | | |
|---|---|---|---|---|---|---|---|---|---|---|
| Component [% b.w.] | a | b | c | d | e | f | g | h | i | j |
| Co-emulsifier such as | 0.50 | 1.00 | 2.00 | 3.00 | 4.00 | 8.00 | 10.00 | 15.00 | 20.00 | 25.00 |
| Caprylyl Dimethicone | | | | | | | | | | |
| EthoxyGlucoside (INCI) | | | | | | | | | | |
| Co-emulsifier such as | 1.00 | 2.00 | 5.00 | 3.00 | 4.00 | 5.00 | 8.00 | 10.00 | 15.00 | 3.00 |
| PEG-6 isostearate | | | | | | | | | | |
| sorbitan (INCI) | | | | | | | | | | |
| Hydrogenated Castor oil | 1.00 | 2.00 | 1.00 | 2.00 | 2.00 | 1.00 | 2.00 | 1.00 | 2.00 | 1.00 |
| and/or microcrystalline | | | | | | | | | | |
| Wax (INCI) | | | | | | | | | | |
| Membrane-forming agent | 3.00 | 5.00 | 1.50 | 2.00 | 3.50 | 3.00 | 5.00 | 1.50 | 2.00 | 3.50 |
| such as phospholipid of | | | | | | | | | | |
| formula (1) as described | | | | | | | | | | |
| in claim 14 | | | | | | | | | | |
| Lipophilic component | 22.00 | 19.00 | 19.00 | 16.50 | 17.50 | 31.00 | 26.00 | 29.00 | 25.00 | 27.50 |
| such as Caprylic/Capric | | | | | | | | | | |
| Triglycerides (INCI) | | | | | | | | | | |
| Water-soluble active | 0.50 | 1.00 | 5.00 | 2.00 | 10.00 | 10.00 | 5.00 | 2.00 | 1.00 | 0.50 |
| ingredient such as | | | | | | | | | | |
| ascorbic acid (INCI) | | | | | | | | | | |
| Antioxidant as defined in | 2.50 | 2.00 | 1.50 | 3.00 | 1.00 | 2.00 | 3.00 | 2.50 | 1.80 | 0.80 |
| claims 7, 8, 9 and 10 of | | | | | | | | | | |
| hereby invention such as | | | | | | | | | | |
| compound of formula A or B | | | | | | | | | | |
| Butylene Glycol | 2.50 | 1.50 | 2.00 | 3.00 | 3.50 | — | — | — | — | — |
| Sorbeth-20 | — | — | — | — | — | 2.50 | 1.50 | 2.00 | 3.00 | 3.50 |
| Water deionized | Qsp | Qsp | Qsp | Qsp | Qsp | Qsp | Qsp | Qsp | Qsp | Qsp |
| | 100 | 100 | 100 | 100 | 100 | 100 | 100 | 100 | 100 | 100 |

### EXAMPLE 4

| | Examples | | | | | | | | | |
|---|---|---|---|---|---|---|---|---|---|---|
| Component [% b.w.] | a | b | c | d | e | f | g | h | i | j |
| Co-emulsifier such as | 0.50 | 1.00 | 2.00 | 3.00 | 4.00 | 8.00 | 10.00 | 15.00 | 20.00 | 25.00 |
| PEG-9 polydimethylsil- | | | | | | | | | | |
| oxyethyl dimethicone (INCI) | | | | | | | | | | |
| Co-emulsifier such as | 1.00 | 2.00 | 5.00 | 3.00 | 4.00 | 5.00 | 8.00 | 10.00 | 15.00 | 3.00 |
| PEG-6 isostearate | | | | | | | | | | |
| sorbitan (INCI) | | | | | | | | | | |
| Hydrogenated Castor oil | 1.00 | 2.00 | 1.00 | 2.00 | 2.00 | 1.00 | 2.00 | 1.00 | 2.00 | 1.00 |
| and/or microcrystalline | | | | | | | | | | |
| Wax (INCI) | | | | | | | | | | |
| Membrane-forming agent | 3.00 | 5.00 | 1.50 | 2.00 | 3.50 | 3.00 | 5.00 | 1.50 | 2.00 | 3.50 |
| such as phospholipid of | | | | | | | | | | |
| formula (1) as described | | | | | | | | | | |
| in claim 14 | | | | | | | | | | |
| Lipophilic component | 22.00 | 19.00 | 19.00 | 16.50 | 17.50 | 31.00 | 26.00 | 29.00 | 25.00 | 27.50 |
| such as Caprylic/Capric | | | | | | | | | | |
| Triglycerides (INCI) | | | | | | | | | | |
| Water-soluble active | 0.50 | 1.00 | 5.00 | 2.00 | 10.00 | 10.00 | 5.00 | 2.00 | 1.00 | 0.50 |
| ingredient such as | | | | | | | | | | |
| ascorbic acid (INCI) | | | | | | | | | | |
| Antioxidant as defined in | 2.50 | 2.00 | 1.50 | 3.00 | 1.00 | 2.00 | 3.00 | 2.50 | 1.80 | 0.80 |
| claims 7, 8, 9 and 10 of | | | | | | | | | | |
| hereby invention such as | | | | | | | | | | |
| compound of formula A or B | | | | | | | | | | |
| Butylene Glycol | 2.50 | 1.50 | 2.00 | 3.00 | 3.50 | — | — | — | — | — |
| Sorbeth-20 | — | — | — | — | — | 2.50 | 1.50 | 2.00 | 3.00 | 3.50 |
| Water deionized | Qsp | Qsp | Qsp | Qsp | Qsp | Qsp | Qsp | Qsp | Qsp | Qsp |
| | 100 | 100 | 100 | 100 | 100 | 100 | 100 | 100 | 100 | 100 |

### EXAMPLE 5

| | Examples | | | | | | | | | |
|---|---|---|---|---|---|---|---|---|---|---|
| Component [% b.w.] | a | b | c | d | e | f | g | h | i | j |
| Co-emulsifier such as | 0.50 | 1.00 | 2.00 | 3.00 | 4.00 | 8.00 | 10.00 | 15.00 | 20.00 | 25.00 |
| C6-8 Alkyl C3-6 Alkyl | | | | | | | | | | |
| Glucoside Dimethicone (INCI) | | | | | | | | | | |
| Co-emulsifier such as | 1.00 | 2.00 | 5.00 | 3.00 | 4.00 | 5.00 | 8.00 | 10.00 | 15.00 | 3.00 |
| Diisostearoyl polyglyceryl-3 | | | | | | | | | | |
| Dilinoleate (INCI) | | | | | | | | | | |
| Hydrogenated Castor oil | 1.00 | 2.00 | 1.00 | 2.00 | 2.00 | 1.00 | 2.00 | 1.00 | 2.00 | 1.00 |
| and/or microcrystalline | | | | | | | | | | |
| Wax (INCI) | | | | | | | | | | |
| Membrane-forming agent | 3.00 | 5.00 | 1.50 | 2.00 | 3.50 | 3.00 | 5.00 | 1.50 | 2.00 | 3.50 |
| such as phospholipid of | | | | | | | | | | |
| formula (1) as described | | | | | | | | | | |
| in claim 14 | | | | | | | | | | |
| Lipophilic component | 22.00 | 19.00 | 19.00 | 16.50 | 17.50 | 31.00 | 26.00 | 29.00 | 25.00 | 27.50 |
| such as Caprylic/Capric | | | | | | | | | | |
| Triglycerides (INCI) | | | | | | | | | | |
| Water-soluble active | 0.50 | 1.00 | 5.00 | 2.00 | 10.00 | 10.00 | 5.00 | 2.00 | 1.00 | 0.50 |
| ingredient such as | | | | | | | | | | |
| ascorbic acid (INCI) | | | | | | | | | | |
| Antioxidant as defined in | 2.50 | 2.00 | 1.50 | 3.00 | 1.00 | 2.00 | 3.00 | 2.50 | 1.80 | 0.80 |
| claims 7, 8, 9 and 10 of | | | | | | | | | | |
| hereby invention such as | | | | | | | | | | |
| compound of formula A or B | | | | | | | | | | |
| Butylene Glycol | 2.50 | 1.50 | 2.00 | 3.00 | 3.50 | — | — | — | — | — |
| Sorbeth-20 | — | — | — | — | — | 2.50 | 1.50 | 2.00 | 3.00 | 3.50 |
| Water deionized | Qsp | Qsp | Qsp | Qsp | Qsp | Qsp | Qsp | Qsp | Qsp | Qsp |
| | 100 | 100 | 100 | 100 | 100 | 100 | 100 | 100 | 100 | 100 |

### EXAMPLE 6

| | Examples | | | | | | | | | |
|---|---|---|---|---|---|---|---|---|---|---|
| Component [% b.w.] | a | b | c | d | e | f | g | h | i | j |
| Co-emulsifier such as | 0.50 | 1.00 | 2.00 | 3.00 | 4.00 | 8.00 | 10 .00 | 15.00 | 20.00 | 25.00 |
| C6-8 Alkyl C3-6 Alkyl | | | | | | | | | | |
| Glucoside Dimethicone (INCI) | | | | | | | | | | |
| Co-emulsifier such as | 1.00 | 2.00 | 5.00 | 3.00 | 4.00 | 5.00 | 8.00 | 10.00 | 15.00 | 3.00 |
| Diisostearoyl polyglyceryl-3 | | | | | | | | | | |
| Dilinoleate (INCI) | | | | | | | | | | |
| Hydrogenated Castor oil | 1.00 | 2.00 | 1.00 | 2.00 | 2.00 | 1.00 | 2.00 | 1.00 | 2.00 | 1.00 |
| and/or microcrystalline | | | | | | | | | | |
| Wax (INCI) | | | | | | | | | | |
| Membrane-forming agent | 3.00 | 5.00 | 1.50 | 2.00 | 3.50 | 3.00 | 5.00 | 1.50 | 2.00 | 3.50 |
| such as phospholipid of | | | | | | | | | | |
| formula (1) as described | | | | | | | | | | |
| in claim 14 | | | | | | | | | | |
| Lipophilic component | 22.00 | 19.00 | 19.00 | 16.50 | 17.50 | 31.00 | 26.00 | 29.00 | 25.00 | 27.50 |
| such as Caprylic/Capric | | | | | | | | | | |
| Triglycerides (INCI) | | | | | | | | | | |
| Water-soluble active | 0.50 | 1.00 | 5.00 | 2.00 | 10.0 0 | 10.00 | 5.00 | 2.00 | 1.00 | 0.50 |
| ingredient such as | | | | | | | | | | |
| ascorbic acid (INCI) | | | | | | | | | | |
| Antioxidant as defined in | 2.50 | 2.00 | 1.50 | 3.00 | 1.00 | 2.00 | 3.00 | 2.50 | 1.80 | 0.80 |
| claims 7, 8, 9 and 10 of | | | | | | | | | | |
| hereby invention such as | | | | | | | | | | |
| compound of formula A or B | | | | | | | | | | |
| Butylene Glycol | 2.50 | 1.50 | 2.00 | 3.00 | 3.50 | — | — | — | — | — |
| Sorbeth-20 | — | — | — | — | — | 2.50 | 1.50 | 2.00 | 3.00 | 3.50 |
| Water deionized | Qsp | Qsp | Qsp | Qsp | Qsp | Qsp | Qsp | Qsp | Qsp | Qsp |
| | 100 | 100 | 100 | 100 | 100 | 100 | 100 | 100 | 100 | 100 |

### EXAMPLE 7

| | Examples | | | | | | | | | |
|---|---|---|---|---|---|---|---|---|---|---|
| Component [% b.w.] | a | b | c | d | e | f | g | h | i | j |
| Co-emulsifier such as | 0.50 | 1.00 | 2.00 | 3.00 | 4.00 | 8.00 | 10.00 | 15.00 | 20.00 | 25.00 |
| Caprylyl Dimethicone | | | | | | | | | | |
| EthoxyGlucoside (INCI) | | | | | | | | | | |
| Co-emulsifier such as | 1.00 | 2.00 | 5.00 | 3.00 | 4.00 | 5.00 | 8.00 | 10.00 | 15.00 | 3.00 |
| polyglyceryl-2 sesquiiso- | | | | | | | | | | |
| stearate(INCI) | | | | | | | | | | |
| Hydrogenated Castor oil | 1.00 | 2.00 | 1.00 | 2.00 | 2.00 | 1.00 | 2.00 | 1.00 | 2.00 | 1.00 |
| and/or microcrystalline | | | | | | | | | | |
| Wax (INCI) | | | | | | | | | | |
| Membrane-forming agent | 3.00 | 5.00 | 1.50 | 2.00 | 3.50 | 3.00 | 5.00 | 1.50 | 2.00 | 3.50 |
| such as phospholipid of | | | | | | | | | | |
| formula (1) as described | | | | | | | | | | |
| in claim 14 | | | | | | | | | | |
| Lipophilic component | 22.00 | 19.00 | 19.00 | 16.50 | 17.50 | 31.00 | 26.00 | 29.00 | 25.00 | 27.50 |
| such as Caprylic/Capric | | | | | | | | | | |
| Triglycerides (INCI) | | | | | | | | | | |
| Water-soluble active | 0.50 | 1.00 | 5.00 | 2.00 | 10.00 | 10.00 | 5.00 | 2.00 | 1.00 | 0.50 |
| ingredient such as | | | | | | | | | | |
| ascorbic acid (INCI) | | | | | | | | | | |
| Antioxidant as defined in | 2.50 | 2.00 | 1.50 | 3.00 | 1.00 | 2.00 | 3.00 | 2.50 | 1.80 | 0.80 |
| claims 7, 8, 9 and 10 of | | | | | | | | | | |
| hereby invention such as | | | | | | | | | | |
| compound of formula A or B | | | | | | | | | | |
| Butylene Glycol | 2.50 | 1.50 | 2.00 | 3.00 | 3.50 | — | — | — | — | — |
| Sorbeth-20 | — | — | — | — | — | 2.50 | 1.50 | 2.00 | 3.00 | 3.50 |
| Water deionized | Qsp | Qsp | Qsp | Qsp | Qsp | Qsp | Qsp | Qsp | Qsp | Qsp |
| | 100 | 100 | 100 | 100 | 100 | 100 | 100 | 100 | 100 | 100 |

### EXAMPLE 8

| | Examples | | | | | | | | | |
|---|---|---|---|---|---|---|---|---|---|---|
| Component [% b.w.] | a | b | c | d | e | f | g | h | i | j |
| Co-emulsifier such as | 0.50 | 1.00 | 2.00 | 3.00 | 4.00 | 8.00 | 10.00 | 15.00 | 20.00 | 25.00 |
| Cetyl Dimethicone | | | | | | | | | | |
| Copolyol (INCI) | | | | | | | | | | |
| Co-emulsifier such as | 1.00 | 2.00 | 5.00 | 3.00 | 4.00 | 5.00 | 8.00 | 10.00 | 15.00 | 3.00 |
| Diisostearoyl polyglyceryl-3 | | | | | | | | | | |
| Dilinoleate (INCI) | | | | | | | | | | |
| Hydrogenated Castor oil | 1.00 | 2.00 | 1.00 | 2.00 | 2.00 | 1.00 | 2.00 | 1.00 | 2.00 | 1.00 |
| and/or microcrystalline | | | | | | | | | | |
| Wax (INCI) | | | | | | | | | | |
| Membrane-forming agent | 3.00 | 5.00 | 1.50 | 2.00 | 3.50 | 3.00 | 5.00 | 1.50 | 2.00 | 3.50 |
| such as phospholipid of | | | | | | | | | | |
| formula (1) as described | | | | | | | | | | |
| in claim 14 | | | | | | | | | | |
| Lipophilic component | 22.00 | 19.00 | 19.00 | 16.50 | 17.50 | 31.00 | 26.00 | 29.00 | 25.00 | 27.50 |
| such as Caprylic/Capric | | | | | | | | | | |
| Triglycerides (INCI) | | | | | | | | | | |
| Water-soluble active | 0.50 | 1.00 | 5.00 | 2.00 | 10.00 | 10.00 | 5.00 | 2.00 | 1.00 | 0.50 |
| ingredient such as | | | | | | | | | | |
| ascorbic acid (INCI) | | | | | | | | | | |
| Antioxidant as defined in | 2.50 | 2.00 | 1.50 | 3.00 | 1.00 | 2.00 | 3.00 | 2.50 | 1.80 | 0.80 |
| claims 7, 8, 9 and 10 of | | | | | | | | | | |
| hereby invention such as | | | | | | | | | | |
| compound of formula A or B | | | | | | | | | | |
| Butylene Glycol | 2.50 | 1.50 | 2.00 | 3.00 | 3.50 | — | — | — | — | — |
| Sorbeth-20 | — | — | — | — | — | 2.50 | 1.50 | 2.00 | 3.00 | 3.50 |
| Water deionized | Qsp | Qsp | Qsp | Qsp | Qsp | Qsp | Qsp | Qsp | Qsp | Qsp |
| | 100 | 100 | 100 | 100 | 100 | 100 | 100 | 100 | 100 | 100 |

### EXAMPLE 9

| | Examples | | | | | | | | | |
|---|---|---|---|---|---|---|---|---|---|---|
| Component [% b.w.] | a | b | c | d | e | f | g | h | i | j |
| Co-emulsifier such as | 0.50 | 1.00 | 2.00 | 3.00 | 4.00 | 8.00 | 10.00 | 15.00 | 20.00 | 25.00 |
| Cetyl Dimethicone | | | | | | | | | | |
| Copolyol (INCI) | | | | | | | | | | |
| Co-emulsifier such as | 1.00 | 2.00 | 5.00 | 3.00 | 4.00 | 5.00 | 8.00 | 10.00 | 15.00 | 3.00 |
| Diisostearoyl polyglyceryl-3 | | | | | | | | | | |
| Dilinoleate (INCI) | | | | | | | | | | |
| Hydrogenated Castor oil | 1.00 | 2.00 | 1.00 | 2.00 | 2.00 | 1.00 | 2.00 | 1.00 | 2.00 | 1.00 |
| and/or microcrystalline | | | | | | | | | | |
| Wax (INCI) | | | | | | | | | | |
| Membrane-forming agent | 3.00 | 5.00 | 1.50 | 2.00 | 3.50 | 3.00 | 5.00 | 1.50 | 2.00 | 3.50 |
| such as phospholipid of | | | | | | | | | | |
| formula (1) as described | | | | | | | | | | |
| in claim 14 | | | | | | | | | | |
| Lipophilic component | 22.00 | 19.00 | 19.00 | 16.50 | 17.50 | 31.00 | 26.00 | 29.00 | 25.00 | 27.50 |
| such as Caprylic/Capric | | | | | | | | | | |
| Triglycerides (INCI) | | | | | | | | | | |
| Water-soluble active | 0.50 | 1.00 | 5.00 | 2.00 | 10.00 | 10.00 | 5.00 | 2.00 | 1.00 | 0.50 |
| ingredient such as | | | | | | | | | | |
| ascorbic acid (INCI) | | | | | | | | | | |
| Antioxidant as defined in | 2.50 | 2.00 | 1.50 | 3.00 | 1.00 | 2.00 | 3.00 | 2.50 | 1.80 | 0.80 |
| claims 7, 8, 9 and 10 of | | | | | | | | | | |
| hereby invention such as | | | | | | | | | | |
| compound of formula A or B | | | | | | | | | | |
| Butylene Glycol | 2.50 | 1.50 | 2.00 | 3.00 | 3.50 | — | — | — | — | — |
| Sorbeth-20 | — | — | — | — | — | 2.50 | 1.50 | 2.00 | 3.00 | 3.50 |
| Water deionized | Qsp | Qsp | Qsp | Qsp | Qsp | Qsp | Qsp | Qsp | Qsp | Qsp |
| | 100 | 100 | 100 | 100 | 100 | 100 | 100 | 100 | 100 | 100 |

### EXAMPLE 10

| | Examples | | | | | | | | | |
|---|---|---|---|---|---|---|---|---|---|---|
| Component [% b.w.] | a | b | c | d | e | f | g | h | i | j |
| Co-emulsifier such as | 0.50 | 1.00 | 2.00 | 3.00 | 4.00 | 8.00 | 10.00 | 15.00 | 20.00 | 25.00 |
| Caprylyl Dimethicone | | | | | | | | | | |
| EthoxyGlucoside (INCI) | | | | | | | | | | |
| Co-emulsifier such as | 1.00 | 2.00 | 5.00 | 3.00 | 4.00 | 5.00 | 8.00 | 10.00 | 15.00 | 3.00 |
| Diisostearoyl polyglyceryl-3 | | | | | | | | | | |
| Dilinoleate (INCI) | | | | | | | | | | |
| Hydrogenated Castor oil | 1.00 | 2.00 | 1.00 | 2.00 | 2.00 | 1.00 | 2.00 | 1.00 | 2.00 | 1.00 |
| and/or microcrystalline | | | | | | | | | | |
| Wax (INCI) | | | | | | | | | | |
| Membrane-forming agent | 3.00 | 5.00 | 1.50 | 2.00 | 3.50 | 3.00 | 5.00 | 1.50 | 2.00 | 3.50 |
| such as phospholipid of | | | | | | | | | | |
| formula (1) as described | | | | | | | | | | |
| in claim 14 | | | | | | | | | | |
| Lipophilic component | 22.00 | 19.00 | 19.00 | 16.50 | 17.50 | 31.00 | 26.00 | 29.00 | 25.00 | 27.50 |
| such as Caprylic/Capric | | | | | | | | | | |
| Triglycerides (INCI) | | | | | | | | | | |
| Water-soluble active | 0.50 | 1.00 | 5.00 | 2.00 | 10.00 | 10.00 | 5.00 | 2.00 | 1.00 | 0.50 |
| ingredient such as | | | | | | | | | | |
| ascorbic acid (INCI) | | | | | | | | | | |
| Antioxidant as defined in | 2.50 | 2.00 | 1.50 | 3.00 | 1.00 | 2.00 | 3.00 | 2.50 | 1.80 | 0.80 |
| claims 7, 8, 9 and 10 of | | | | | | | | | | |
| hereby invention such as | | | | | | | | | | |
| compound of formula A or B | | | | | | | | | | |
| Butylene Glycol | 2.50 | 1.50 | 2.00 | 3.00 | 3.50 | — | — | — | — | — |
| Sorbeth-20 | — | — | — | — | — | 2.50 | 1.50 | 2.00 | 3.00 | 3.50 |
| Water deionized | Qsp | Qsp | Qsp | Qsp | Qsp | Qsp | Qsp | Qsp | Qsp | Qsp |
| | 100 | 100 | 100 | 100 | 100 | 100 | 100 | 100 | 100 | 100 |

### EXAMPLE 11

| | Examples | | | | | | | | | |
|---|---|---|---|---|---|---|---|---|---|---|
| Component [% b.w.] | a | b | c | d | e | f | g | h | i | j |
| Co-emulsifier such as | 0.50 | 1.00 | 2.00 | 3.00 | 4.00 | 8.00 | 10.00 | 15.00 | 20.00 | 25.00 |
| Caprylyl Dimethicone | | | | | | | | | | |
| EthoxyGlucoside (INCI) | | | | | | | | | | |
| Co-emulsifier such as | 1.00 | 2.00 | 5.00 | 3.00 | 4.00 | 5.00 | 8.00 | 10.00 | 15.00 | 3.00 |
| PEG-6 isostearate | | | | | | | | | | |
| sorbitan (INCI) | | | | | | | | | | |
| Hydrogenated Castor oil | 1.00 | 2.00 | 1.00 | 2.00 | 2.00 | 1.00 | 2.00 | 1.00 | 2.00 | 1.00 |
| and/or microcrystalline | | | | | | | | | | |
| Wax (INCI) | | | | | | | | | | |
| Membrane-forming agent | 3.00 | 5.00 | 1.50 | 2.00 | 3.50 | 3.00 | 5.00 | 1.50 | 2.00 | 3.50 |
| such as phospholipid of | | | | | | | | | | |
| formula (1) as described | | | | | | | | | | |
| in claim 10 | | | | | | | | | | |
| Lipophilic component | 22.00 | 19.00 | 19.00 | 16.50 | 17.50 | 31.00 | 26.00 | 29.00 | 25.00 | 27.50 |
| such as Caprylic/Capric | | | | | | | | | | |
| Triglycerides (INCI) | | | | | | | | | | |
| Water-soluble active | 0.50 | 1.00 | 5.00 | 2.00 | 10.00 | 10.00 | 5.00 | 2.00 | 1.00 | 0.50 |
| ingredient such as | | | | | | | | | | |
| ascorbic acid (INCI) | | | | | | | | | | |
| Antioxidant as defined in | 2.50 | 2.00 | 1.50 | 3.00 | 1.00 | 2.00 | 3.00 | 2.50 | 1.80 | 0.80 |
| claims 7, 8, 9 and 10 of | | | | | | | | | | |
| hereby invention such as | | | | | | | | | | |
| compound of formula A or B | | | | | | | | | | |
| Butylene Glycol | 2.50 | 1.50 | 2.00 | 3.00 | 3.50 | — | — | — | — | — |
| Sorbeth-20 | — | — | — | — | — | 2.50 | 1.50 | 2.00 | 3.00 | 3.50 |
| Water deionized | Qsp | Qsp | Qsp | Qsp | Qsp | Qsp | Qsp | Qsp | Qsp | Qsp |
| | 100 | 100 | 100 | 100 | 100 | 100 | 100 | 100 | 100 | 100 |

### EXAMPLE 12

| | Examples | | | | | | | | | |
|---|---|---|---|---|---|---|---|---|---|---|
| Component [% b.w.] | a | b | c | d | e | f | g | h | i | j |
| Co-emulsifier such as | 0.50 | 1.00 | 2.00 | 3.00 | 4.00 | 8.00 | 10.00 | 15.00 | 20.00 | 25.00 |
| Caprylyl Dimethicone | | | | | | | | | | |
| EthoxyGlucoside (INCI) | | | | | | | | | | |
| Co-emulsifier such as | 1.00 | 2.00 | 5.00 | 3.00 | 4.00 | 5.00 | 8.00 | 10.00 | 15.00 | 3.00 |
| polyglyceryl-2 sesquiiso- | | | | | | | | | | |
| stearate (INCI) | | | | | | | | | | |
| Hydrogenated Castor oil | 1.00 | 2.00 | 1.00 | 2.00 | 2.00 | 1.00 | 2.00 | 1.00 | 2.00 | 1.00 |
| and/or microcrystalline | | | | | | | | | | |
| Wax (INCI) | | | | | | | | | | |
| Membrane-forming agent | 3.00 | 5.00 | 1.50 | 2.00 | 3.50 | 3.00 | 5.00 | 1.50 | 2.00 | 3.50 |
| such as phospholipid of | | | | | | | | | | |
| formula (1) as described | | | | | | | | | | |
| in claim 14 | | | | | | | | | | |
| Lipophilic component | 22.00 | 19.00 | 19.00 | 16.50 | 17.50 | 31.00 | 26.00 | 29.00 | 25.00 | 27.50 |
| such as Caprylic/Capric | | | | | | | | | | |
| Triglycerides (INCI) | | | | | | | | | | |
| Water-soluble active | 0.50 | 1.00 | 5.00 | 2.00 | 10.00 | 10.00 | 5.00 | 2.00 | 1.00 | 0.50 |
| ingredient such as | | | | | | | | | | |
| ascorbic acid (INCI) | | | | | | | | | | |
| Antioxidant as defined in | 2.50 | 2.00 | 1.50 | 3.00 | 1.00 | 2.00 | 3.00 | 2.50 | 1.80 | 0.80 |
| claims 7, 8, 9 and 10 of | | | | | | | | | | |
| hereby invention such as | | | | | | | | | | |
| compound of formula A or B | | | | | | | | | | |
| Butylene Glycol | 2.50 | 1.50 | 2.00 | 3.00 | 3.50 | — | — | — | — | — |
| Sorbeth-20 | — | — | — | — | — | 2.50 | 1.50 | 2.00 | 3.00 | 3.50 |
| Water deionized | Qsp | Qsp | Qsp | Qsp | Qsp | Qsp | Qsp | Qsp | Qsp | Qsp |
| | 100 | 100 | 100 | 100 | 100 | 100 | 100 | 100 | 100 | 100 |

### EXAMPLE 13

| | Examples | | | | | | | | | |
|---|---|---|---|---|---|---|---|---|---|---|
| Component [% b.w.] | a | b | c | d | e | f | g | h | i | j |
| Co-emulsifier such as | 0.50 | 1.00 | 2.00 | 3.00 | 4.00 | 8.00 | 10.00 | 15.00 | 20.00 | 25.00 |
| C6-8 Alkyl C3-6 Alkyl | | | | | | | | | | |
| Glucoside Dimethicone (INCI) | | | | | | | | | | |
| Co-emulsifier such as | 1.00 | 2.00 | 5.00 | 3.00 | 4.00 | 5.00 | 8.00 | 10.00 | 15.00 | 3.00 |
| Polyglyceryl-10 penta- | | | | | | | | | | |
| hydroxystearate (INCI) | | | | | | | | | | |
| Hydrogenated Castor oil | 1.00 | 2.00 | 1.00 | 2.00 | 2.00 | 1.00 | 2.00 | 1.00 | 2.00 | 1.00 |
| and/or microcrystalline | | | | | | | | | | |
| Wax (INCI) | | | | | | | | | | |
| Membrane-forming agent | 3.00 | 5.00 | 1.50 | 2.00 | 3.50 | 3.00 | 5.00 | 1.50 | 2.00 | 3.50 |
| such as phospholipid of | | | | | | | | | | |
| formula (1) as described | | | | | | | | | | |
| in claim 14 | | | | | | | | | | |
| Lipophilic component | 22.00 | 19.00 | 19.00 | 16.50 | 17.50 | 31.00 | 26.00 | 29.00 | 25.00 | 27.50 |
| such as Caprylic/Capric | | | | | | | | | | |
| Triglycerides (INCI) | | | | | | | | | | |
| Water-soluble active | 0.50 | 1.00 | 5.00 | 2.00 | 10.00 | 10.00 | 5.00 | 2.00 | 1.00 | 0.50 |
| ingredient such as | | | | | | | | | | |
| ascorbic acid (INCI) | | | | | | | | | | |
| Antioxidant as defined in | 2.50 | 2.00 | 1.50 | 3.00 | 1.00 | 2.00 | 3.00 | 2.50 | 1.80 | 0.80 |
| claims 7, 8, 9 and 10 of | | | | | | | | | | |
| hereby invention such as | | | | | | | | | | |
| compound of formula A or B | | | | | | | | | | |
| Butylene Glycol | 2.50 | 1.50 | 2.00 | 3.00 | 3.50 | — | — | — | — | — |
| Sorbeth-20 | — | — | — | — | — | 2.50 | 1.50 | 2.00 | 3.00 | 3.50 |
| Water deionized | Qsp | Qsp | Qsp | Qsp | Qsp | Qsp | Qsp | Qsp | Qsp | Qsp |
| | 100 | 100 | 100 | 100 | 100 | 100 | 100 | 100 | 100 | 100 |

### EXAMPLE 14

| | Examples | | | | | | | | | |
|---|---|---|---|---|---|---|---|---|---|---|
| Component [% b.w.] | a | b | c | d | e | f | g | h | i | j |
| Co-emulsifier such as | 0.50 | 1.00 | 2.00 | 3.00 | 4.00 | 8.00 | 10.00 | 15.00 | 20.00 | 25.00 |
| Caprylyl Dimethicone | | | | | | | | | | |
| EthoxyGlucoside (INCI) | | | | | | | | | | |
| Co-emulsifier such as | 1.00 | 2.00 | 5.00 | 3.00 | 4.00 | 5.00 | 8.00 | 10.00 | 15.00 | 3.00 |
| polyglyceryl-2 sesquiiso- | | | | | | | | | | |
| stearate (INCI) | | | | | | | | | | |
| Hydrogenated Castor oil | 1.00 | 2.00 | 1.00 | 2.00 | 2.00 | 1.00 | 2.00 | 1.00 | 2.00 | 1.00 |
| and/or microcrystalline | | | | | | | | | | |
| Wax (INCI) | | | | | | | | | | |
| Membrane-forming agent | 3.00 | 5.00 | 1.50 | 2.00 | 3.50 | 3.00 | 5.00 | 1.50 | 2.00 | 3.50 |
| such as phospholipid of | | | | | | | | | | |
| formula (1) as described | | | | | | | | | | |
| in claim 14 | | | | | | | | | | |
| Lipophilic component | 22.00 | 19.00 | 19.00 | 16.50 | 17.50 | 31.00 | 26.00 | 29.00 | 25.00 | 27.50 |
| such as Caprylic/Capric | | | | | | | | | | |
| Triglycerides (INCI) | | | | | | | | | | |
| Water-soluble active | 0.50 | 1.00 | 5.00 | 2.00 | 10.00 | 10.00 | 5.00 | 2.00 | 1.00 | 0.50 |
| ingredient such as | | | | | | | | | | |
| ascorbic acid (INCI) | | | | | | | | | | |
| Antioxidant as defined in | 2.50 | 2.00 | 1.50 | 3.00 | 1.00 | 2.00 | 3.00 | 2.50 | 1.80 | 0.80 |
| claims 7, 8, 9 and 10 of | | | | | | | | | | |
| hereby invention such as | | | | | | | | | | |
| compound of formula A or B | | | | | | | | | | |
| Butylene Glycol | 2.50 | 1.50 | 2.00 | 3.00 | 3.50 | — | — | — | — | — |
| Sorbeth-20 | — | — | — | — | — | 2.50 | 1.50 | 2.00 | 3.00 | 3.50 |
| Water deionized | Qsp | Qsp | Qsp | Qsp | Qsp | Qsp | Qsp | Qsp | Qsp | Qsp |
| | 100 | 100 | 100 | 100 | 100 | 100 | 100 | 100 | 100 | 100 |

### EXAMPLE 15

| | Examples | | | | | | | | | |
|---|---|---|---|---|---|---|---|---|---|---|
| Component [% b.w.] | a | b | c | d | e | f | g | h | i | j |
| Co-emulsifier such as | 1.90 | — | — | 4.00 | 6.00 | 6.00 | — | 5.00 | 5.00 | — |
| polysorbate 80 | | | | | | | | | | |
| Co-emulsifier such as | — | 1.90 | — | — | — | | 5.00 | — | — | 5.00 |
| polysorbate 20 | | | | | | | | | | |
| Co-emulsifier such as | — | — | 1.90 | — | — | — | — | — | — | — |
| polysorbate 60 | | | | | | | | | | |
| Co-emulsifier such as | 7.70 | — | — | 10.00 | 9.00 | 9.00 | — | 15.00 | 15.00 | — |
| Sorbitan oleate | | | | | | | | | | |
| Co-emulsifier such as | — | 7.70 | — | — | — | — | — | — | — | — |
| Sorbitan laurate | | | | | | | | | | |
| Co-emulsifier such as | — | — | 7.70 | — | — | — | 15.00 | — | — | 15.00 |
| Sorbitan stearate | | | | | | | | | | |
| Ethanol | 4.80 | 4.80 | 4.80 | 5.00 | 5.00 | 5.00 | 5.00 | 5.00 | 5.00 | 5.00 |
| Antioxidant as defined in | 2.50 | 2.00 | 1.50 | 3.00 | 1.00 | 2.00 | 3.00 | 2.50 | 1.80 | 0.80 |
| claims 7, 8, 9 and 10 of | | | | | | | | | | |
| hereby invention such as | | | | | | | | | | |
| compound of formula A or B | | | | | | | | | | |
| Membrane-forming agent | 4.80 | 4.80 | 4.80 | 5.00 | 5.00 | 5.00 | 5.00 | 5.00 | 5.00 | 5.00 |
| such as phospholipid of | | | | | | | | | | |
| formula (1) as described | | | | | | | | | | |
| in claim 14 | | | | | | | | | | |
| Lipophilic component | 76.80 | 76.80 | 76.80 | 72.00 | — | 70.00 | 70.00 | — | 60.00 | 60.00 |
| such as Caprylic/Capric | | | | | | | | | | |
| Triglycerides (INCI) | | | | | | | | | | |
| Lipophilic component | — | — | — | — | 70.00 | — | — | 60.00 | — | — |
| such as hydrogenated | | | | | | | | | | |
| Polydecenes (INCI) | | | | | | | | | | |
| Water-soluble active | 0.80 | 0.80 | 0.80 | 0.80 | 1.00 | 1.00 | 1.00 | 2.00 | 2.00 | 2.00 |
| ingredient such as | | | | | | | | | | |
| ascorbic acid (INCI) | | | | | | | | | | |
| Water deionized | 3.20 | 3.20 | 3.20 | 3.20 | 4.00 | 4.00 | 4.00 | 8.00 | 8.00 | 8.00 |

### EXAMPLE 16

| | Examples | | | | | | | | | | |
|---|---|---|---|---|---|---|---|---|---|---|---|
| Component [% b.w.] | a | b | c | d | e | f | g | h | i | j | k |
| Cetyl PEG/PPG-10/1 | 10.00 | 10.00 | 10.00 | — | 25.00 | — | 25.00 | 20.00 | — | 20.00 | 20.00 |
| Dimethicone | | | | | | | | | | | |
| Bis-PEG/PPG-20/20 | — | — | — | 10.00 | — | 25.00 | — | — | 20.00 | — | — |
| Dimethicone | | | | | | | | | | | |
| Co-emulsifier such as | 17.50 | | — | 17.50 | 6.00 | — | — | 6.00 | — | — | 17.50 |
| polysorbate 80 | | | | | | | | | | | |
| Co-emulsifier such as | — | 17.50 | — | — | — | 6.00 | — | — | 6.00 | — | — |
| polysorbate 20 | | | | | | | | | | | |
| Co-emulsifier such as | — | — | 17.50 | — | — | — | 6.00 | — | — | 6.00 | — |
| polysorbate 60 | | | | | | | | | | | |
| Co-emulsifier such as | 10.10 | — | — | 10.10 | 6.00 | — | — | 6.00 | — | — | 10.10 |
| Sorbitan oleate | | | | | | | | | | | |
| Co-emulsifier such as | — | 10.10 | — | — | — | 6.00 | — | — | 6.00 | — | — |
| Sorbitan laurate | | | | | | | | | | | |
| Co-emulsifier such as | — | — | 10.10 | — | — | — | 6.00 | — | — | 6.00 | — |
| Sorbitan stearate | | | | | | | | | | | |
| Ethanol | 5.10 | 5.110 | 5.10 | 4.00 | 7.00 | 7.00 | 7.00 | 8.00 | 8.00 | 8.00 | 5.10 |
| Antioxidant as defined in | 2.50 | 2.00 | 1.50 | 3.00 | 1.00 | 2.00 | 3.00 | 2.50 | 1.80 | 0.80 | 0.95 |
| claims 7, 8, 9 and 10 of | | | | | | | | | | | |
| hereby invention such as | | | | | | | | | | | |
| compound of formula A or B | | | | | | | | | | | |
| Membrane-forming agent | 5.10 | 5.10 | 5.10 | 4.00 | 5.00 | 5.00 | 5.00 | 5.00 | 5.00 | 5.00 | 5.10 |
| such as phospholipid of | | | | | | | | | | | |
| formula (1) as described | | | | | | | | | | | |
| in claim 14 | | | | | | | | | | | |
| Lipophilic component | 30.1 | 30.1 | 30.1 | — | 15.00 | — | 15.00 | 20.00 | — | 20.00 | 28.90 |
| such as Caprylic/Capric | | | | | | | | | | | |
| Triglycerides (INCI) | | | | | | | | | | | |
| Lipophilic component | — | — | — | 30.00 | — | 15.00 | — | — | 20.00 | — | — |
| such as hydrogenated | | | | | | | | | | | |
| Polydecenes (INCI) | | | | | | | | | | | |
| Water-soluble active | 2.40 | 2.40 | 2.40 | 3.00 | 6.00 | 6.00 | 6.00 | 10.00 | 10.00 | 10.00 | 3.10 |
| ingredient such as | | | | | | | | | | | |
| ascorbic acid (INCI) | | | | | | | | | | | |
| Water deionized | 9.60 | 9.60 | 9.60 | 15.00 | 20.00 | 20.00 | 20.00 | 25.00 | 25.00 | 25.00 | 9.30 |

1. A method of improving the stability of water-soluble ingredients in a cosmetic formula against degradation/decomposition which is induced by oxygenous environment and water comprising a nanodispersion, which nanodispersion comprises
(a) a membrane-forming molecule,
(b) a coemulsifier,
(c) a lipophilic component, and
(d) a water-soluble ingredient sensitive to oxidation
in cosmetic end formulations, the nanodispersion being obtained by (α) mixing the components (a), (b) and (c) until a homogeneous liquid is obtained (a nanodispersion pre-phase), and (β) adding a water or aqueous phase (a discontinuous phase) which contains the water-soluble ingredient and optionally a non-aqueous polar organic solvent to the liquid obtained in step (α).

2. The method according to claim 1 wherein component (d) is ascorbic acid or vitamin C.

3. The method according to claim 1, wherein the non-aqueous polar organic solvents are polyols, homopolymeric or block copolymeric liquid ethers.

4. The method according to claim 1, wherein step (α) is carried out in anhydrous medium.

5. The method according to claim 1, wherein step (β) is carried out without homogenisation.

6. The method according to claim 1, wherein the particles in the nanodispersion have an average diameter of <100 nm.

7. The method according to claim 1, comprising additionally a component (e) which is an antioxidant.

8. The method according to claim 7, wherein component (e) is selected from the group consisting of carbon bridged hindered phenols, ester bridged hindered phenols, amide bridged hindered phenols, lactones of hindered phenols, sterically hindered oxylamines and sterically hindered hydroxylamines.

9. The method according to claim 7, wherein component (e) is a compound of the formula A or B 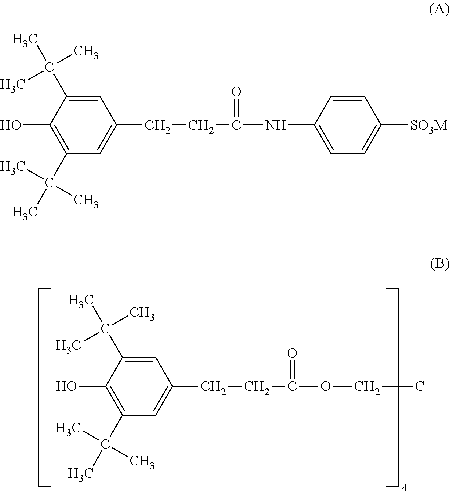
wherein M is hydrogen or sodium.

10. The method according to claim 7, comprising further hydrophilic or lipophilic antioxidants selected from the group consisting of tocopherols and its ester of acids, tocotrienol and its ester of acids, ascorbic acid esters, retinoids, carotenoids, glutathione peroxidise, catalase, superoxide dismutase, ubiquinone and idebenone, lipoic acid and its derivatives, rutinic acid and its derivatives, phenolic acids, flavonoids and polyphenols, chlorogenic acid and ferulic acid.

11. The method according to claim 7, wherein the weight ratio between component (e) and component (d) is from 1:100 to 10:1.

12. The method according to claim 1, wherein component (a) is a substance which is suitable for forming so-called bilayers, component (b) is a substance which preferably forms W/O structures, and component (c) is a functional lipophilic active agent customarily used in cosmetics.

13. The method according to claim 1, wherein component (a) is a phospholipid, a hydrated or partially hydrated phospholipid, a lysophospholipid, a ceramide or mixtures thereof.

14. The method according to claim 1, wherein component (a) is a phospholipid of the formula 1 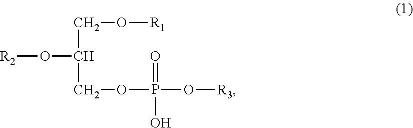
wherein
R₁ is C₁₀-C₂₀acyl;
R₂ is hydrogen or C₁₀-C₂₀acyl,
R₃ is hydrogen, 2-trimethylamino-1-ethyl (or referred as lecithin), 2-amino-1-ethyl; C₁-C₅alkyl which is unsubstituted or substituted by one or several carboxy, hydroxy or amino groups; the inositol or glyceryl group;
or salts of these compounds of the formula 1.

15. The method according to claim 1, wherein component (a) is present in the nanodispersion in a concentration of 0.1 to 30% by weight, based on the total weight of components (a), (b), (c) and (d).

16. The method according to claim 1, wherein component (b) is a siloxane or organosiloxane, a sorbitan derivative or a mixture of these substances.

17. The method according to claim 16, wherein component (b) is a compound of the formula 2 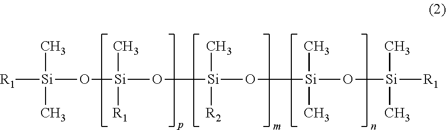
wherein
m is a number from 1 to 20,
n is a number from 0 to 500,
p is a number from 0 to 50,
R₁ is C₁-C₃₀alkyl or phenyl,
R₂ is —C_{c}H_{2c}—(OC₂H₄)ₐ—(OC₃H₆)_{b}—(OC₄H₈)_{d}—R₃,
R₃ is hydrogen, hydroxy, linear or branched C₁-C₁₂alkyl, linear or branched C₁-C₆alkoxy, linear or branched C₂-C₁₂acyloxy, —NHCH₂CH₂COOM, —NHCO(CH₂)_{d}—COOM, C₁-C₃₀-carboxyacyl, —NHCO(CH₂)_{d}—OH or —NH₃Y
M is hydrogen, sodium, potassium, lithium, ammonium or an organic ammonium,
Y is a monovalent organic or inorganic anion such as chloro, bromo, sulfate or carboxylate,
a is a number from 0 to 100,
b is a number from 0 to 50,
c is a number from 0 to 5, and
d is a number from 0 to 10.

18. The method according to claim 1, wherein component (b) is present in the nanodispersion in a concentration of 1 to 50% by weight, based on the total weight of the components (a), (b), (c) and (d).

19. The method according to claim 1, wherein component (c) is a natural or synthetic or partially synthetic di- or triglyceride, a mineral oil, silicone oil, wax, fatty alcohol, guerbet alcohol or the ester thereof, or a mixture of these substances.

20. The method according to claim 1, wherein component (c) comprises esters represented by the class RCO—OR′, wherein RCO— represents the carboxylic acid radical and —OR′ is the alcohol residue; glyceryl esters and derivatives, alkoxylated alcohol esters, alkoxylated alcohol glyceryl esters and derivatives, alkoxylated alcohol ethers, fats and oils, ethers, hydrocarbons or silicones, or a mixture of these substances.

21. The method according to claim 1, wherein component (c) is present in the cosmetic end formulation in a concentration of 0.1 to 80% by weight based on the total weight of the components (a), (b), (c) and (d).

22. The method according to claim 1, comprising additionally a component (f) which is a non-aqueous polar organic solvent to fasten and improve the hydration rate of component (a).

23. The method according to claim 1, wherein the nanodispersion is present in the end formulation in a concentration of 0.01 to 99% by weight, based on the total weight of the cosmetic end-product.

24. The method according to claim 1, wherein at least one component (a), (b) or (c) is an ingredient used in cosmetics for treating or protecting the skin, mucosae or hair.

25. A cosmetic end formulation in the form of a stick, which comprises the nanodispersion prepared by the method as defined in claim 1.

26. A cosmetic end formulation in the form of a gel, which comprises the nanodispersion prepared by the method as defined in claim 1.

27. A cosmetic end formulation in the form of a cream, lotion or milk, which comprises the nanodispersion prepared by the method as defined in claim 1.

28. A cosmetic end formulation in the form of a spray or aerosol, which comprises the nanodispersion prepared by the method as defined in claim 1.

29. A cosmetic end formulation in the form of a foam, which comprises the nanodispersion as prepared by the method as defined in claim 1.

30. A cosmetic end formulation in the form of a paste, which comprises the nanodispersion as prepared by the method as defined in claim 1.

31. A cosmetic end formulation in the form of a powder, lacquer, pellet or makeup, which comprises the nanodispersion as prepared by the method as defined in claim 1, the nanodispersion being present in dehydrated form.
